(19) United States
(12) Patent Application Publication
(10) Pub. No.: US 2024/0052129 A1
(43) Pub. Date: Feb. 15, 2024

(54) A VERSATILE METHOD TO VALORIZE CELLULOSIC WASTE TEXTILES

(71) Applicant: SHARETEX AB, Bjärred (SE)

(72) Inventors: Lars STIGSSON, BJARRED (SE); Miguel SANCHIS SEBASTIA, ARSTA (SE)

(21) Appl. No.: 18/270,766

(22) PCT Filed: Dec. 17, 2021
(86) PCT No.: PCT/SE2021/051280

§ 371 Date: Jul. 3, 2023

(30) Foreign Application Priority Data
Jan. 4, 2021 (SE) 2150003-8
Feb. 15, 2021 (SE) 2150166-3
Jun. 1, 2021 (SE) 2150696-9

(51) Int. Cl. C08J 11/08; C12P 19/02; D01F 2/28

(52) U.S. Cl. CPC C08J 11/08; C12P 19/02; D01F 2/28; C08J 2301/02; C08J 2367/00

(57) ABSTRACT

A process for valorization of a crude waste textile material comprising cotton fibers, viscose fibers and/or other cellulosic textile fibers into cellulose pulp. The process comprises the steps of: —providing a comminuted textile material comprising cotton fibers, viscose fibers and/or other cellulosic fibers; —mixing the comminuted textile material with at least one solvent in the form of an aqueous metal halide (MeX), comprising MeX in a concentration in the range of 60 to 80 wt %, or in the form of a metal halide hydrate having the formula MeX·n(H₂O), wherein the metal Me is Li⁺, Zn²⁺, Mg²⁺, or Ca²⁺, wherein the halide X is Cl⁻ (chlorine) or Br⁻ (bromine), and wherein the integer “n” is 2 to 6, such as 3 or 4; —heating the mixture of the comminuted textile material and the metal halide to dissolve, at least partly, the comminuted textile material to provide a solution of cellulose and MeX; and —precipitating cellulose pulp from the solution of cellulose and MeX, preferably by adding an aqueous solution to the solution of cellulose and MeX to precipitate cellulose pulp; —recovering cellulose pulp.

### FIELD OF THE INVENTION

The present invention relates to a process for valorization of crude waste textile material by converting it into cellulose pulp. The process comprises increasing the reactivity of waste cellulosic textiles and production of cellulose pulp. The cellulose pulp can inter alia be used in spinning cellulosic fibers, derivatized to provide cellulose derivatives or polymers derived from cellulose, or converted to monosaccharides in high yield to serve as starting material in the production of various chemicals.

### DESCRIPTION OF RELATED ART

Waste Textiles

Around 90 million tons of textile fibers were produced in 2014 and the market is expected to grow steadily over the next few years, exceeding well over 120 million tons in 2025. This enlargement of the textile sector poses an environmental challenge generating large amounts of waste textiles. It is estimated that less than 10% of all used textile products are recycled today. Currently, landfilling and incineration are the most common techniques for managing this waste.

In line with the principles of circular economy, it will be desirable to develop new valorization strategies that can recover and recycle textile fibers so that this resource can be reintroduced to the market and consumers at a higher value than that of incineration (or landfill).

There are processes available for fiber recovery from waste textiles comprising cellulosic fibers, which can be favorable from a circular perspective (such as re-use in the second-hand market) and for regeneration of new textiles where the fibers remain in the material system. However, the cellulosic polymers building up the textile tend to be depolymerized during use and washing, and not all recycled material is suitable for recycling. Colorants, and other additives in the textile material may affect downstream valorization negatively (decreased solubility in solvents, catalyst poison, microbial enzyme inhibition, etc.). Moreover, certain manufactured cellulosic fiber materials, such as viscose and recently commercialized fibers using alkali as solvent, have polymers with low molecular weight, which is further lowered during use.

Therefore, there is a need for new valorization technologies and climate effective processes such that crude waste textile material comprising cellulosic fibers, independent of wear and tear, can at least partly be used as a feedstock for more valuable products.

Cotton

Cotton is typically used to provide cotton fibers. Cotton fibers are a type of commonly used cellulosic textile fiber. Cotton fibers are used to make a number of textile products. These include terrycloth for highly absorbent bath towels and robes, denim for blue jeans, cambric, popularly used in the manufacture of blue work shirts (from which the term “blue-collar” originates), corduroy, seersucker, and cotton twill. Socks, underwear, and most T-shirts are made from cotton. Bed sheets often are made from cotton. Cotton is also used to make yarn used in crochet and knitting. Fabric also can be made from recycled or recovered cotton that otherwise would be thrown away during the spinning, weaving, or cutting process.

Although many fabrics are made completely of cotton, some materials blend cotton with other fibers, including rayon and synthetic fibers such as polyester. It can either be used in knitted or woven fabrics, as it can be blended with elastane to make a stretchier thread for knitted fabrics, and apparel such as stretch jeans. Cotton can also be blended with linen producing fabrics with the benefits of both materials. Linen-cotton blends are wrinkle resistant, retain heat more effectively than only linen, and are thinner, stronger, and lighter than only cotton.

In addition to the textile industry, cotton is inter alia used in fishing nets, coffee filters, tents, explosives manufacture (e.g. nitrocellulose), cotton paper, and bookbinding.

Cotton production is in the range of 25 million tons per year, and it is estimated that several hundred million tons of used cotton products are due for recycle over the next several years.

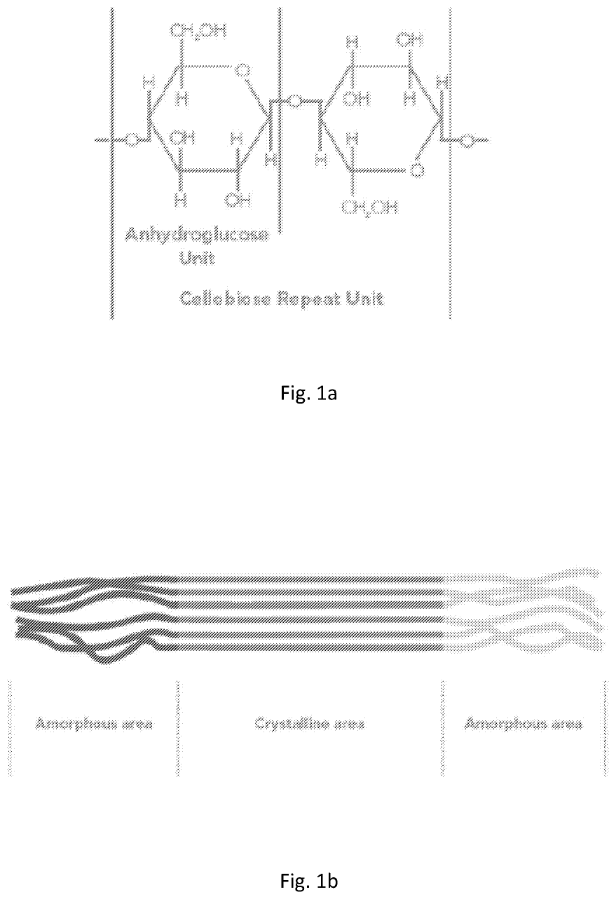

After scouring and bleaching, cotton fibers contain 99% cellulose. Cellulose is a macromolecule—a polymer made up of a long chain of glucose molecules linked by C-1 to C-4 oxygen bridges with elimination of water (glycoside bonds). The anhydro glucose units are linked together as beta-cellobiose; therefore, anhydro-beta-cellobiose is the repeating unit of the polymer chain (FIG. 1a). The average number of repeating units linked together to form the cellulose polymer is referred to as the “degree of polymerization”, or DP.

The cellulose chains within cotton fibers tend to develop molecular interactions with each other, both through hydrogen bonds and hydrophobic interactions. These hydrogen bonds occur between the hydroxyl groups of adjacent molecules (intermolecular) as well as the adjacent hydroxyl groups within the same molecule (intramolecular) and are most prevalent between the parallel, well-ordered molecules in the crystalline regions of the fiber. The crystalline region of a fiber is shown in FIG. 1b.

Cotton fibers have one of the highest molecular weight and structural order among all plant fibers. This means that cellulose present in cotton mainly differs from cellulose present in wood by having a higher degree of polymerization and crystallinity (Table 1). This means that the cellulose molecules are well-ordered and tightly stacked with each other, which is associated with higher fiber strength.

TABLE 1
Degree of polymerization and crystallinity
of different cellulose fibers

| | Average Degree | Average |
|---|---|---|
| Fiber | of Polymerization* | crystallinity (%)** |
| Cotton | 9,000-15,000 | 73 |
| Viscose rayon | 250-450 | 25-40 |
| Avicel/MCC*** | | 80-85 |
| Cold alkali fiber | 150-250 | 50-70 |
| Carbamate fiber | — | 35-45 |
| Wood pulp | 600-1,500 | 35-40 |

*Joseph, M., Introduction to Textile Science, 5^{th} Edition, 1986.
**Shirley Institute; measured by X-ray diffraction.
***Microcrystalline cellulose (MCC)

The three hydroxyl groups, one primary and two secondaries, in each repeating cellobiose unit of cellulose are chemically reactive. These groups can undergo substitution reactions in procedures designed to modify the cellulose fibers or in the application of dyes and finishes for crosslinking. The hydroxyl groups also serve as principal sorption sites for water molecules. Directly absorbed water is firmly chemisorbed on the cellulosic hydroxyl groups by hydrogen bonding.

Of particular interest in the case of cellulosic fibers, such as cotton or viscose rayon fibers, is the response of their strength to variations in moisture content. In the case of regenerated and derivative cellulose fibers, strength generally decreases with increasing moisture content. In contrast, the strength of cotton generally increases with increased moisture. This difference among fibers in their response to moisture is explained in terms of intermolecular hydrogen bonding between cellulose chains and their degree of crystallinity.

Man-Made Cellulose Fibers

Apart from cotton, there are other cellulosic fibers present in the textile industry, both natural and regenerated. Regenerated fibers are man-made fibers based on polymers that are obtained through chemical processing of natural materials and the most popular regenerated cellulose fibers are viscose and lyocell. In fact, regenerated cellulose fibers is one of the market segments growing the fastest in the textile industry, as their consumption increased 7.5% in 2019 while the overall consumption of textile fibers increased only 3.4% during the same period. Thus, it could be expected that the share of regenerated cellulose fibers present in waste textiles will increase over the next years.

Viscose is the regenerated cellulose fiber most used in textile fibers and it is produced by dissolving the cellulose (usually extracted from wood pulp) in an alkaline solution, followed by derivatization to cellulose xanthate and subsequent regeneration of the cellulose solution. Another example of this type of fiber is lyocell, whose production is based on the dissolution of cellulose without derivatizing the polymer. Such a dissolution requires the use of an organic solvent called N-methyl-morpholine-N-oxide (NMMO).

There has recently been an interest in cold alkali fiber processes and their resulting fibers, which are based on dissolving cellulose in a cold aqueous alkaline solution and spinning the fibers directly from this solution (see e.g. WO 2020/171767 A1). The production of these fibers requires a starting material with a low degree of polymerization (typically 150-250) and a more detailed description of their current state of the art can be found in the paper “Cellulose in NaOH-water based solvents: a review” by Tatiana Budtova and Patrick Navard (Cellulose, Springer Verlag, 2016, 23 (1), pp. 5-55, doi 10.1007/s10570-015-0779-8).

Cellulose Allomorphs, Crystallinity and Degree of Polymerization

The properties of the cellulose contained in waste textiles are far from homogenous due to the presence of many different types of fabric in this residue. Perhaps the most important difference is that natural occurring cellulose contains mostly the allomorph cellulose I while man-made cellulose products contain primarily the allomorph cellulose II. This means that natural fibers, such as cotton, do not have the same cellulose structure as man-made cellulose fibers, such as viscose or cold alkali fibers. Apart from this, cotton fibers exhibit higher degree of polymerization and crystallinity than man-made cellulose fibers and therefore they behave differently in recycling processes. For example, cellulose II is more difficult to dissolve in cold alkali than cellulose I. This implies that recycling processes need to be versatile in order to tolerate this heterogeneity and be able to accept the different types of fabric.

The main difference in the morphology of cellulose I and II is that the cellulose chains are parallel in the former while they are antiparallel in the latter. The parallelism in cellulose I allows to establish stronger hydrophobic interactions between the cellulose chains, which leads to a tighter stacking of the structure with a smaller spacing between the cellulose sheets. Moreover, cellulose II can expand its structure to intercalate two water molecules in the crystal lattice, leading to an inflated structure with low density. All this implies that cellulose II exhibits a crystal structure more accessible than cellulose I and therefore existing technologies need to be redesigned and optimized to handle the higher accessibility associated with cellulose II.

Apart from an increased accessibility, cellulose II is usually associated with a lower degree of polymerization because the manufacturing processes of cellulose products tend to decrease the DP of the material. For example, cotton fibers have a DP in the range 9000-15000 while viscose fibers have a DP in the range 250-450 (Table 1). The reduction of DP during cellulose processing has not been traditionally a concern due to the relatively high DP of the starting material. However, such a large reduction in DP would not be acceptable when processing a starting material with low DP and therefore existing technologies need to be redesigned and optimized to maintain the DP of the material to a larger extent in order to handle regenerated cellulose waste fibers.

### SUMMARY

An objective of the present invention is to provide a method for valorization of waste or recycled textiles into valuable organic platform chemicals that in turn can be used for manufacturing of new textiles or other consumer products.

In a first aspect, there is provided a process for valorization of a crude waste textile material comprising cotton fibers, viscose fibers and/or other cellulosic textile fibers, such as cold alkali fibers, into cellulose pulp. The process comprises the steps of providing a comminuted textile material comprising cotton fibers, viscose fibers and/or other cellulosic fibers, mixing the comminuted textile material with at least one solvent in the form of an aqueous metal halide (MeX). The aqueous metal halide (MeX) comprises MeX in a concentration in the range of 60 to 80 wt %, or is in the form of a metal halide hydrate having the formula MeX·n(H₂O). If the aqueous metal halide has the formula MeX·n(H₂O), the metal Me is Li⁺, Zn²⁺, Mg²⁺, or Ca²⁺, and the halide X is Cl⁻ (chlorine) or Br⁻ (bromine), and the integer “n” is 2 to 6, such as 3 or 4. Preferably, the metal Me is Li⁺ or Zn²⁺, such as being Zn²⁺. The process further comprises a step of heating the mixture of the comminuted textile material and the metal halide to dissolve, at least partly, the comminuted textile material to provide a solution of cellulose and MeX, and precipitating cellulose pulp from the solution of cellulose and MeX. Preferably, cellulose pulp is precipitated by adding an aqueous solution to the solution of cellulose and MeX to precipitate cellulose pulp. The precipitated cellulose of the cellulose pulp has higher reactivity than the cellulose of waste textile material. The process further comprises a step of recovering a cellulose pulp.

This process is advantageous in that it allows for the transformation of cellulose-based waste textiles into a pure cellulose pulp. As opposed to other recycling technologies, this process enables the recycling of cellulose-based textiles several times. Further, the MeX solution dissolves the cellulose contained in the textiles, which is then regenerated from the solution. The regenerated cellulose has a higher reactivity than the original material, making it more amenable to further processing or conversion to the targeted products. The cellulose pulp is easier to re-dissolve and easier to process into, for example, cellulose derivatives. Furthermore, once the cellulose pulp has been precipitated, it can easily be separated from the solvent, i.e. the metal halide (MeX).

Further, the recovered cellulose pulp may serve as a starting material for the formation of various regenerated products. The crude waste textile material is therefore an inexpensive (and sustainable) source of cellulose.

According to some embodiments, the process only reduces the degree of polymerization (DP) of the regenerated cellulose to a small extent. The minimal reduction in DP facilitates the use of the regenerated cellulose in the production of new textile fibers several times, creating a cascading effect that keeps the material within the fashion loop as long as possible. For example, regenerated cellulose from cotton might be directly spun into new fibers or used in the production of viscose fabrics and, in turn, regenerated cellulose from viscose could be used in cold alkali fiber spinning.

According to other embodiments, parameters such as temperature and reaction time of the process are selected such that the process reduces the degree of polymerization (DP) of the regenerated cellulose. A reduction in DP may be desired in cases where the cellulose contained in the material cannot be valorized as a polymer anymore. In such cases, depolymerization to short-chain cellulose or glucose would be more favorable to valorize the material via production of fine chemicals and other industrial intermediates.

In one embodiment, MeX is ZnCl₂·3(H₂O), ZnCl₂·4(H₂O), ZnBr₂·3(H₂O), ZnBr₂·4(H₂O) or a mixture of two or more of these. Preferably, MeX is ZnCl₂·4(H₂O). Zn compounds are, due to their low toxicity and specific interactions with cellulose, advantageous for use in this process. Zinc chloride hydrate is an acidic solvent due to both the Lewis acidity of the Zn²⁺ and the Brønsted acidity of the coordinated water, which can not only dissolve but also partially break down cellulose.

In another embodiment, MeX is ZnCl₂·4(H₂O), and the process preferably further comprises a step of recovering purified MeX as ZnCl₂·4(H₂O) and recycling it to the step of mixing the comminuted textile material with aqueous MeX. As cellulose is precipitated as cellulose pulp, aqueous MeX can simply be filtered off. Further, MeX remaining in the cellulose pulp can be extracted by washing the pulp, not only providing purer cellulose pulp, but also recovering MeX.

In yet another embodiment, the solvent is an aqueous metal halide (MeX) comprising MeX in a concentration in the range of 65 to 75 wt %. Preferably, MeX is ZnCl₂, ZnBr₂, or a mixture thereof. MeX can only effectively dissolve cellulose when the hydroxyl groups of cellulose complete the first hydration shell of the cation and participate in the formation of the second hydration shell. Thus, concentrations below 65% are not very effective because the second hydration shell is complete without the hydroxyl groups of cellulose, whereas concentrations above 75% are not very effective because the second hydration shell remains incomplete.

The process may further comprise restoring the solvent and recycling at least a portion of said restored solvent comprising MeX to said step of mixing the comminuted textile material with MeX. This is beneficial since the MeX can be reused, thus providing a more environmentally friendly and cost-efficient process. Restoring the solvent comprises restoring the concentration of MeX, e.g. concentrating the MeX if it has been diluted. Further, it may comprise purifying contaminants.

In one embodiment, the step of restoring, e.g. concentrating, the solvent, comprises purifying MeX by at least one of evaporation, crystallization, and extraction. MeX may be ZnCl₂, ZnCl₂ being restored to and recycled as ZnCl₂·4(H₂O).

The step of restoring the MeX solvent may further comprise removing heavy metals, such as by washing solid MeX with water. Preferably, the resulting aqueous solution comprising heavy metals is depleted of heavy metals, e.g. by membrane filtration, evaporation or precipitation. Preferably, at least part of the aqueous solution depleted of heavy metals is used to form part of the aqueous solution in the step of precipitating cellulose pulp.

According to an embodiment, the process further comprises a step of deriving cellulose of type II from the MeX solvent. The cellulose of type II may be derived as nanocellulose. Further, or alternatively, it may be derived as microcrystalline cellulose (MCC). The cellulose of type II is typically derived before restoring the solvent. Further, the process may comprise a step of deriving cellulose of type II, such as nanocellulose and/or microcrystalline cellulose (MCC), from an aqueous solution used to wash the precipitated pulp. In an embodiment wherein cellulose of type II is to be derived, at least 50 wt %. such as at least 75 or 90 wt %, of the cellulose in comminuted textile material may be cellulose of type II. Thus, the waste textile material may comprise at least 50 wt %. such as at least 75 or 90 wt %, viscose fibers, lyocell fibers, and/or fibers cold alkali type textile fibers. Further, the waste textile material may comprise less than 10 wt % cotton fibers, such as less than 5 wt % cotton fibers.

In one embodiment, the step of heating the mixture of the comminuted textile material and the metal halide is performed at a temperature in the range of 50° C. to 170° C. Preferably, the step of heating the mixture of the comminuted textile material and the metal halide is performed in a temperature range of 60° C. to 80° C. Further, the mixture of the comminuted textile material and the metal halide is typically heated during a time range of 3 minutes to 4 hours, such as for 15 minutes to 2 hours. The temperature and time range are selected and controlled depending on the composition of crude waste textile material. The selected temperature and time range are beneficial since they are typically sufficient to dissolve more than 90% of the cellulosics.

Further, the combination of residence time and temperature in the heating step may be chosen based on the target DP of the cellulose pulp, as well as the composition of the crude waste textile material. For example, targeting a cellulose pulp with similar DP to the starting material is typically associated with lower temperatures and/or shorter residence times whereas targeting a cellulose pulp with lower DP than the starting material is typically associated with higher temperatures and/or longer residence times. Further, the residence time and temperature in the heating step may also be varied depending on the composition of waste textiles because these conditions need to be adapted to the special characteristics of cellulose II. Usually, it is preferred to lower the temperature and/or shorten the residence time to account for a higher accessibility and lower DP of the material. For example, cotton waste fibers may be treated at higher temperature, e.g. 70 to 100° C., such as 75 to 85° C., for a longer time, e.g. 1 to 2 h, whereas regenerated cellulose waste fibers may be treated at lower temperature, e.g. 50 to 80° C., such as 60 to 70° C., for a shorter time, e.g. 15 to 45 min, to achieve a similar reduction of DP in the resulting cellulose material.

The crude waste textile material may be comminuted by at least one of shredding, grinding and milling, or a combination thereof, to provide a comminuted textile material. The crude waste textile may be comminuted by milling. Optionally, MeX is added to be present in the milling step. Ball milling of the crude waste textile material also tends to depolymerize the cellulose, which is beneficial as it facilitates the subsequent dissolving thereof. Hence, the decrease in DP is due to a combined effect of ball milling and the MeX treatment in the mixing and heating steps.

In yet another embodiment, any fraction of polyester present in the crude waste textile material is, at least partly, separated prior to adding the aqueous solution to precipitate the cellulose pulp. In this case, the polyester fraction is preferably separated by dissolving the polyester in a solvent and separating the solid cellulosic textile fibers by a solid liquid separation process prior to mixing the cellulosic fibers with MeX. In addition or alternatively, a solid polyester fraction is separated from the solution of cellulose and MeX by a solid liquid separation process after having dissolved the textile fibers to provide a solution of cellulose and MeX, but before precipitating pulp. This facilitates a true circular economy for the crude waste textile material. Mechanical separation is preferred for mixed textiles (e.g. polyester textiles mixed with cotton textiles) while chemical separation is preferred for mixed fabrics (e.g. polycotton). Mechanical separation does not allow efficient separation of the polyester from the crude waste textile material since they are often too intimately linked, e.g. in the form of polycotton. Still, an initial sorting step may be beneficial to enrich cellulose textiles, e.g. by removing synthetic textiles, such as polyester fabrics. The chemical routes on the other hand are selective, and can thus separate polyester even when intimately embedded with e.g. cotton in the crude waste textile material.

In one embodiment, the crude waste textile material is subjected to a sorting step prior to mixing the comminuted textile material with at least one solvent in the form of an aqueous metal halide (MeX). The sorting step serves to remove metal pieces, such as buttons, rivets, and/or zippers, remove plastic pieces, such as buttons and/or zippers, and enrich a given type of textile fibers, such as viscose fibers or cotton fibers. Such a mechanical sorting step is an efficient way to remove features such as metal pieces, such as buttons, rivets, and/or zippers, remove plastic pieces, such as buttons and/or zippers, and facilitates the process downstream of the sorting step.

In a further embodiment, the average degree of polymerization (DP) of cellulose in the cellulosic textile fibers in the crude waste textile material to be mixed with the solvent is lower than 700. Preferably, the crude waste textile material comprises viscose fiber. More preferably, the crude waste textile material comprises at least 50 wt % viscose fiber, such as at least 75 wt % viscose fiber.

The average degree of polymerization (DP) of cellulose in the cellulosic textile fibers in the crude waste textile material to be mixed with the solvent may be lower than 500, and/or the crude waste textile material may comprise at least 50 wt %, such as at least 75 wt %, cellulosic textile fibers in which the cellulose has an average degree of polymerization (DP) of less than 400. The crude waste textile material may comprise cold alkali fibers. More preferably, the crude waste textile material comprises at least 50 wt % cold alkali fibers, such as at least 75 wt % cold alkali fibers.

Further, an acid, such as hydrochloric acid, may be added in the step of heating the mixture of the comminuted textile material and the metal halide to reduce the average degree of polymerization (DP) and thus reduce and control the average degree of polymerization (DP) of the cellulose pulp.

In some embodiments, the crude waste textile material is processed in a manner avoiding any substantial de-polymerization of the cellulose. Thus, the average degree of polymerization (DP) of the resulting cellulose pulp may be 80 to 100% of the average degree of polymerization (DP) of the cellulose in the cellulosic textile fibers in the crude waste textile material. According to such an embodiment, the average degree of polymerization (DP) of the cellulose in the cellulosic textile fibers in the crude waste textile material is typically 700 or lower, and/or the crude waste textile material comprises viscose rayon fibers and/or cold alkali type of textile fibers. For cellulosic textile fibers in the crude waste textile material having a high DP, it may be difficult to completely avoid de-polymerization. Further, in processing fibers with very high DP (e.g. 700) it might be difficult to operate the process to completely avoid depolymerization. In such embodiments, a reduction in DP of up to 20% could thus be seen as acceptable.

Further, the average degree of polymerization (DP) of the resulting cellulose pulp may be 80 to 200, or 400 to 600. Such pulp may inter alia be useful to regenerate textile fibers in the form of cold alkali fibers and in the form of viscose fibers, respectively.

The intrinsic viscosity of aqueous cellulose is related to the chain length and weight properties of the cellulose and may be used to calculate the degree of polymerization (DP) of the material. The average degree of polymerization (DP) of e.g. the cellulose in the cellulosic textile fibers in the crude waste textile material and/or the resulting cellulose pulp may thus be determined by determining its intrinsic viscosity.

According to an embodiment, the average degree of polymerization (DP) is calculated using the IV method, where the limiting viscosity number of the raw material and dissolving pulp is determined by dissolving the materials in 0.5M cupriethylenediamine solution and measuring the limiting viscosity in a capillary-tube viscometer, according to ISO-5351:2010. The average degree of polymerization is then estimated through the following equations (1) and (2), in which η is the viscosity:

DP^{0.9}=1.65·η (Evans & Wallins, 1989) (1)

DP^{0.905}=0.75·η (Sihtola et al., 1963) (2)

The Evans & Wallins equation (1) is an update of the Sihtola et al. equation (2). Equation (1) is applied within a DP range of (700<DP<5000), whereas the Sihtola equation (2) is applied outside this range.

In one embodiment the cellulose pulp is precipitated by adding an aqueous solution comprising an alcohol, e.g. methanol, ethanol or propanol, a ketone, e.g. acetone, and/or adding an aqueous solution comprising at least 80 wt %, such as at least 90 or 95 wt %, water. Preferably, the cellulose pulp is precipitated adding an aqueous solution comprising at 95 wt %, such as at least 99 wt %, water.

In another embodiment the recovered cellulose pulp is subject to a washing step to remove impurities, such as metals and inorganic salts, and/or a bleaching step. Preferably, the ISO brightness of the bleached cellulose pulp according to ISO 3688:1999 exceeds 90. This is advantageous in that the recovered cellulose pulp is purified from contaminants, and discoloration is also removed by the bleaching.

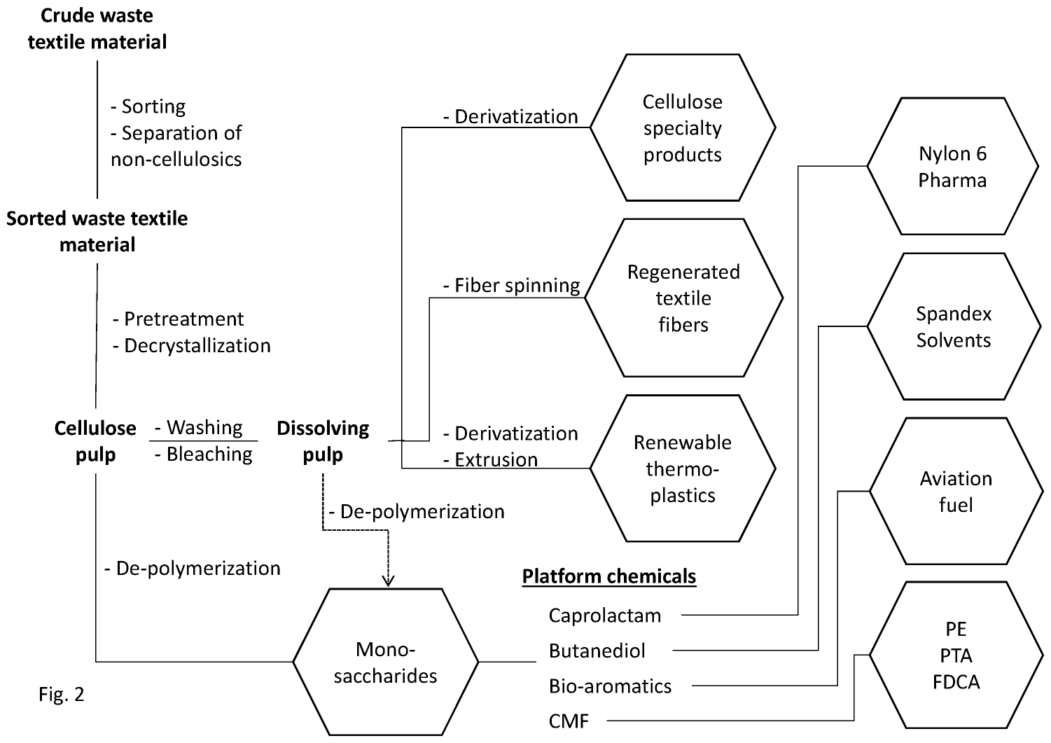

The recovered cellulose pulp may be used as a starting material to provide platform chemicals. This is of interest, especially if the average degree of polymerization (DP) in the cellulose pulp is low, such as lower than 500, lower than 400 or lower than 250. According to embodiments in which the recovered cellulose pulp is to be saccharified to monosaccharides by acid hydrolysis and/or by enzyme treatment, the crude waste textile material comprises cellulose fibers having low degree of polymerization (DP), e.g. viscose fibers and/or other cellulosic textile fibers, such as cold alkali fibers. Preferably, the crude waste textile material comprises at least 50 wt % viscose fibers and/or other cellulosic textile fibers, such as cold alkali fibers. In order to provide a feedstock for production of various platform chemicals, such as the ones listed in FIG. 2, the recovered cellulose pulp may be saccharified to monosaccharides by acid hydrolysis and/or by enzyme treatment. Saccharification of the recovered cellulose pulp by hydrolysis or by enzymes is greatly facilitated using the present process, and the saccharification can be performed with recyclable solid acids with a minor addition of homogeneous acids only, saving energy and reducing the amount of solid and liquid waste. The de-polymerization conducted by the saccharification provides monosaccharides, which may in turn be converted into a number of platform chemicals which can be used as starting materials for forming additional regenerated materials. Such platform chemicals include e.g. caprolactam, butanediol, bio-aromatics and 5-chloromethylfurfural (CMF).

According to an embodiment, the recovered cellulose pulp may be used as starting material to provide chloromethylfurfural (CMF). In such an embodiment, recovered cellulose pulp may be saccharified to monosaccharides by acid hydrolysis and converted to CMF in the same step, i.e. without firstly isolating and optionally purifying formed monosaccharides. As disclosed herein below, CMF can be prepared from carbohydrates, i.e. cellulose, by treatment with a hydrochloric acid, such as concentrated hydrochloric acid, in an organic solvent. Typical conditions comprise treatment at a temperature of 80 to 180° C. for 0.5 to 40 hrs.

The acid hydrolysis may be performed in the presence of one or more solid acids, sulphonic acids, or mineral acids, such as hydrochloric acid or sulfuric acid, or a combination thereof.

In one embodiment the monosaccharides are converted to organic fine chemicals by fermentation, one or more microbial processes and/or catalytic processes, or a combination thereof. The monosaccharides may be converted to 5-chloromethylfurfural (CMF), caprolactam, 5-(hydroxymethyl)furfural (HMF), 2,5-furandicarboxylic acid (FDCA), or adipic acid. This is beneficial since advantageous compounds such as CMF, HMF, FDCA or adipic acid can be obtained. CMF can in turn be further transformed into e.g. polyethylene (PE), para-xylene, terephthalic acid (PTA), and polyethylene terephthalate (PET), as well as numerous commodity and specialty chemicals through its derivatives.

In one embodiment, the monosaccharides obtained from saccharification of the cellulose pulp are converted into bio-aromatics, which in turn may be converted, with processes known in the art, into e.g. additives for (renewable) aviation fuel.

In one embodiment, the monosaccharides obtained from saccharification of the cellulose pulp are converted into butanediol, which in turn may be converted, with processes known in the art, into e.g. spandex or solvents.

In one embodiment, the monosaccharides obtained from saccharification of the cellulose pulp are converted into caprolactam, which in turn may be converted, with processes known in the art, into e.g. polyamide 6 (Nylon 6). Alternatively, caprolactam may be used in the synthesis of pharmaceutical drugs and/or in the pharma industry.

In another embodiment, platform organic chemicals such as ethanol, butanediol and caprolactam (caprolactone over HMF) are produced from glucose by a well-known catalytic or microbial process, such as fermentation.

One or more acids used in the acid hydrolysis and/or one or more enzymes used in the enzyme treatment may be at least partially recycled to be reused for further treatment of cellulose pulp. This provides a more cost-efficient and sustainable process.

The cellulose pulp may further be used as dissolving pulp in the production of regenerated cellulosic fibers, especially if the average degree of polymerization (DP) is high, such as above 500. This is advantageous since the recovered cellulose pulp acts as a starting material for forming regenerated cellulosic fibers, i.e. regenerated textile fibers, e.g. viscose fibers. Such fibers may be produced using, for instance, fiber spinning. The dissolving pulp has preferably been subjected to a bleaching and/or a washing step before the dissolving pulp is used to produce regenerated cellulosic fibers.

In another embodiment, the cellulose pulp is used as dissolving pulp in the production of renewable thermoplastics, especially if the average degree of polymerization (DP) is high, such as above 500. This is advantageous since the recovered cellulose pulp acts as a starting material for forming renewable thermoplastics. Similarly to when producing regenerated cellulosic fibers, the dissolving pulp has preferably been subjected to a bleaching and/or a washing step before the dissolving pulp is used to produce renewable thermoplastics. To obtain the renewable thermoplastics, the dissolving pulp may derivatized or subjected to extrusion in a manner known in the art.

In one embodiment the process further comprises the step of derivatizing the cellulose in the cellulose pulp into carboxymethyl cellulose, cellulose acetate or cellulose ethers. The process may further comprise the step of derivatizing the cellulose in the cellulose pulp into cellulose acetate. Preferably, cellulose is derivatized into cellulose acetate under homogeneous conditions wherein cellulose is dissolved in an ionic liquid. The obtained cellulose acetate may be used in the spinning of a textile fiber.

According to an alternative embodiment, cellulose acetate is formed by adding at least one of acetic acid and acetic acid anhydride to the solution of cellulose and MeX and subsequently precipitating cellulose acetate.

In one embodiment, the process further comprises the step of converting the cellulose in the cellulose pulp into dialcohol cellulose.

In a further embodiment the process for valorization of a crude waste textile material is integrated into a pulping process at a pulp mill, such as a pulp mill designed for production of kraft, chemithermomechanical pulp CTMP/CMP, thermomechanical pulp, or a mill operating with recycled cellulosic fiber as feedstock.

### DETAILED DESCRIPTION

Overview Possible Routes for Valorization of a Crude Waste Textile Material

In FIG. 2, an overview of the present process for valorization of a crude waste textile material according to a further exemplary embodiment is provided.

The present process is based on a dissolution step that allows the transformation of cellulose-based waste textiles into a pure cellulose dissolving pulp. As opposed to other recycling technologies, the present process allows to recycle cellulose-based textiles several times. In addition, an end-of-life strategy has been developed to valorize the textiles after the fibers can no longer be recycled. Thus, the present process completely closes the fashion loop, contributing towards the creation of a circular economy.

Upstream the present process, waste textiles are collected and sorted based on their composition. Cellulose-based fibers, such as cotton or viscose, can be separated from each other as well as from synthetic fibers and other types of fibers through near-infrared methods. These methods are currently applied at some demonstration facilities in Sweden, for example Siptex in Malmb or Wargon Innovation in Vargon. After sorting, the fractions containing 100% cotton and 100% viscose are the preferred feedstocks for further processing, even though the present process can be adapted to treat polycotton (i.e. cotton mixed with synthetic fibers) as well.

Foreign elements, such as seams, buttons, zippers, etc., are removed from the sorted textiles, which are subsequently exposed to a metal halide solution, such as a ZnCl₂ solution. This solution dissolves the cellulose contained in the textiles, which can be regenerated from the solution through the addition of an appropriate antisolvent (e.g. water, ethanol or acetone).

The regenerated cellulose has a higher reactivity than the original material, which makes it more amenable to further processing or conversion to the targeted products, while reducing its degree of polymerization (DP) only to a small extent. The minimal reduction in DP allows the use of the regenerated cellulose in the production of new textile fibers several times, creating a cascading effect that keeps the material within the fashion loop as much as possible. For example, regenerated cellulose from cotton might be directly spun into new fibers or used in the production of viscose fabrics and, in turn, regenerated cellulose from viscose could be used in the production of cold alkali solvent type fibers.

A portion of the dissolving pulp can be subtracted at each stage of the textile fiber cascade to produce different types of chemicals. This strategy allows the exploitation of the DP of the dissolving pulp to produce chemicals that cannot be obtained from glucose or other platform chemicals where the fibers have been completely depolymerized. For example, regenerated cellulose from cotton could be used to produce cellulose ethers and esters, while regenerated cellulose from viscose could be used to produce cellulose acetate, a very attractive product due to its thermoplastic nature and usability in textile materials. Thus, the dissolution step provides the possibility of producing specialty chemicals from green sources, apart from keeping the textile fibers longer within the fashion loop.

Once the DP of the pulp is insufficient to produce new textile fibers, or driven by economic considerations, the dissolving pulp can be completely depolymerized via acid or enzymatic hydrolysis. This depolymerization can be performed with high efficiency thanks to the high reactivity of the dissolving pulp. The product of the depolymerization is typically a monosaccharide, which in turn can be converted into a platform chemical that can be used in the manufacturing of a myriad of products. For example, the monosaccharide glucose can be fermented into 1,4-butanediol or caprolactam, while 5-chloromethylfurfural can be converted into bioplastics or bio-aromatics. Some of these platform chemicals can be used to manufacture synthetic fibers, such as elastane or nylon 6, and therefore even our end-of-life strategy provides the possibility of keeping the value of the material within the fashion industry.

Crude Waste Textile Material

The fashion industry has already started to show an interest in fiber recycling to reduce the environmental impact associated with waste textiles. Even though there are no commercial-scale recycling processes available, some small-scale projects have been initiated to recycle waste textiles into fibers that can be spun again. However, textile fibers cannot be recycled indefinitely because their properties (water absorption, tensile strength, etc.) degrade with each recycling loop, and therefore end-of-life valorization techniques will be needed for fibers that have already been recycled several times. Moreover, certain textiles have already a low molecular weight cellulose chain (corresponding to an intrinsic viscosity IV lower than about 600 mL/g as determined by using ISO5351:2010) in the virgin garment such as viscose, carbamate, and other cold alkali fiber textiles, and such textile fibers cannot be efficiently recycled or regenerated into new textile fibers. In this context, organic chemical production provides an attractive alternative to valorize such waste textile material.

For ideal waste management, the textiles should be reused rather than recycled and recycled rather than discarded. However, textile fibers become damaged over time. After having been recycled, the fibers become shorter and the degree of polymerization decreases, hindering the possibility of mechanically or chemically creating new fibers and fabric from the material. It is mainly this material that is intended for conversion to, for example, CMF (Chloromethyl furfural) or glucose in accordance with the present disclosure. These used textiles, low quality and degraded cotton, or other cellulosic fibers such as viscose and cold alkali fibers, are characterized by having cellulose polymers with an intrinsic viscosity (IV) of less than about 600 mL/g.

Preferably, the crude waste textile material comprising cotton fibers to be processed have been subjected to a sorting step prior to being processed in the present process. The sorting step may identify and separate garments made of pure cotton, polyester, acrylic, wool, polyamide and man-made cellulose fibers as well as fiber blends thereof. Buttons, seams, zippers and other non-process elements may be removed from the sorted waste textiles in this step as well.

There are several facilities worldwide that are dedicated to the collection and sorting of waste textiles. For example, Wargön Innovation in Vargön and Siptex in Malmö. The separation is typically based on fingerprint analysis with near infrared technology and the process is automated through artificial intelligence and machine learning.

The cellulose polymers that are building blocks in the cellulosic textile fibers of the crude waste textile material, such as viscose and cotton waste textiles, used in the present process, may have a large fraction, preferably over 50% by weight, of polymers with an average intrinsic viscosity lower than about 600 mL/g as determined by ISO 5351:2010. In line with this, according to one embodiment, the cellulosic textile fibers of the crude waste textile material charged to the process comprise a large fraction, preferably over 50% by weight of waste textiles, of cotton fibers, viscose fibers, and/or cold alkali fibers, having an average cellulosic polymer molecular chain length lower than corresponding to an intrinsic viscosity (IV) of 600 mL/g as determined by ISO 5351:2010.

One example of the present disclosure is based on using sorted crude waste textile material substantially comprising at least one of viscose rayon fibers and cold alkali fibers, such as conventional cold alkali fiber or carbamate fiber. Cold alkali fibers are described in Cellulose in “NaOH-water based solvents: a review” by Tatiana Budtova and Patrick Navard (Cellulose, Springer Verlag, 2016, 23 (1), pp. 5-55, doi 10.1007/s10570-015-0779-8).Crude waste textile material where the average intrinsic viscosity of the cellulose polymers in the waste textile feed stream is lower than about 300 mL/g, as determined by using ISO5351:2010, can advantageously be converted to valuable organic chemicals in accordance with the present disclosure. Alternatively, they can be used as feedstock for preparation of regenerated textile fibers in the form of viscose, and/or cold alkali fibers.

The sorted waste textiles are subjected to shredding and/or any other mechanical treatment (e.g. shredding, milling, grinding, refining, etc.) necessary to ensure the feedability of the material in the subsequent bleaching steps. Ball milling has a high influence on the microscopic and macroscopic properties of the resulting material after treatment, such as structure, morphology, crystallinity, and thermal stability. When treating microcrystalline cellulose derived from cotton linters in a planetary ball mill at 200 rpm for 4-8 hours in dry and wet conditions with three solvents (water, toluene, 1-butanol), one observes de-structuring of the cellulose during the process (particularly for water), leading to variations in morphology and crystallinity. These differences are purportedly attributed to the effect of the solvent on the hydrogen bonding amongst the cellulose particles. In dry conditions, aggregated globular particles (5-10 μm diameter) are formed from the cotton fabric, with a lower degree of crystallinity. The crystalline structure is to a high extent converted to cellulose II, which is much more accessible for the MeX treatment in the present invention.

Viscose and other lower crystallinity cellulosic fibers fed to the process of the present invention may not need to be ball milled prior to treatment with the MeX cellulose solvent.

In one embodiment MeX solvent is charged to be present during treatment of waste textiles in a milling environment.

Bleaching

As disclosed in WO 2021/006798 A1, pulping processes include bleaching steps to remove essentially all of the residual lignin from the cellulose and to increase brightness of the cellulose pulp and may involve bleaching agents such as oxygen, chlorine dioxide and hydrogen peroxide. Modern pulp mills often have an alkaline oxygen delignification stage as a first delignification or bleaching step downstream of the digesters. In a kraft mill, the alkali used in the oxygen delignification step may be oxidized white liquor wherein the sodium sulfide in white liquor is oxidized to thiosulfate or sulfate. Spent alkali from this step may be used for washing the brownstock and may be added to the kraft chemicals recovery cycle for recovery and recycling of sodium and sulfur compounds. Bleaching of chemical pulps to reach the desired degree of lignin content and pulp brightness is frequently composed of four or more discrete steps.

According to an embodiment, the recovered cellulose pulp is introduced upstream of any of these bleaching stages in accordance with the present disclosure and subject to a pulping bleaching step.

Pulp brightness is defined as the amount of incident light reflected from paper under specified conditions, usually reported as the percentage of light reflected, so a higher number means a brighter or whiter paper. The international community uses ISO standards.

In one embodiment the waste textile material is bleached prior to the MeX step.

In one embodiment the pulp recovered from the MeX stage is directly or indirectly bleached with at least one bleaching agent.

Bleaching herein can be performed with one or several bleaching compounds well known in the art of bleaching cellulosic material. The material can be bleached in a total chlorine free (TCF) or an elemental chlorine free (ECF) sequence to remove the different dyes present in the material. Examples of treatments typically included in a TCF sequence are oxygen (O), peroxide (P), peracetic acid (Paa), ozone (Z) and hydrosulfite while an ECF sequence usually includes a chlorine dioxide treatment (D). It is preferred that the design of the bleaching sequence matches that of an existing kraft or sulfite mill, so that the process can be integrated in existing facilities to minimize the capital and operational costs. For example, the waste effluents from the bleaching sequence could be handled within the current operations of the mill, typically via concentration (evaporation, membrane filtration, etc.) and incineration of the remaining BOD and COD in the kraft or MgO boiler (depending on the type of mill).

Bleaching of waste cellulosic textile with bleaching agents in accordance with this disclosure, such as bleaching performed prior to or after the MeX stage, such as, for example, bleaching with zinc dithionite, the resulting effluents therefrom can advantageously be integrated with the MeX chemicals recovery loop of the present invention or the chemicals recovery system of a kraft or sulphite pulp mill.

MeX Treatment of Waste Cellulosics

The objective of an MeX treatment step where comminuted textile material is mixed with MeX is to dissolve the cellulosic fibers, e.g. cotton or viscose fibers, and increase their reactivity at conditions controlling the depolymerization of the polysaccharides. Shorter chain oligosaccharides, cellobiose and glucose may not be desired, but can still be products from the MeX treatment stage by selecting harsher process conditions.

Once the cellulose textile material is dissolved, the cellulosic fibers may be precipitated to provide a cellulose pulp with higher reactivity. By dissolving the cellulosic fibers, they are precipitated in a more reactive form being easier to re-dissolve and easier to process into, for example, cellulose derivatives. Thus, the production of cellulose pulp in accordance with the present invention provides for a new and effective route to prepare reactive cellulose dissolving pulp. The cellulose pulp, e.g. dissolving pulp, may be used in a variety of applications as outlined herein.

The molten metal salt hydrates (MeX) that are preferred to use as cellulose solvents in the MeX step of present disclosure are ZnCl₂·4H₂O, ZnBr₂·4H₂O, LiBr·4H₂O, MgCl₂ and CaCl₂, alone or in mixtures. The concentration of solvent should be in the range of 60-80%, preferably in the range of 65-75%.

Dissolution of the cellulosic substrate in MeX can depolymerize the cellulose fibers to various extent depending on the treatment conditions. In some embodiments, it is preferred to maintain the DP of the material as high as possible and therefore the treatment conditions are selected so that the average DP of the pulp may be in the range 80-100% of the average DP of the starting material, preferably in the range 90-100%. In other embodiments, it is preferred to lower the DP of the material to a desired value usually in the range 20-70% of the average DP of the starting material, preferably in the range 40-60%.

According to an embodiment, the average degree of polymerization (DP) of the resulting cellulose pulp is 400 to 600. Such pulp may inter alia be useful to regenerate textile fibers in the form of viscose fibers.

According to an embodiment, the average degree of polymerization (DP) of the resulting cellulose pulp is 80 to 200. Such pulp may inter alia be useful to regenerate textile fibers in the form of cold alkali fibers.

Dissolution of the cellulosic substrate in MeX can decrease the crystallinity of the cellulose fibers, especially at harsh treatment conditions. Thus, harsh treatments (associated with lowering the DP) tend to yield a cellulose pulp with lower crystallinity than the starting material while mild treatments (associated with maintaining the DP) tend to yield a cellulose pulp with similar crystallinity than the starting material. This means that, apart from DP, the present invention allows to deliver cellulose pulp with varying crystallinity depending on the requirements imposed on the pulp.

Typically, the MeX and comminuted waste textile materials are charged directly or transferred from the comminuting stage to the MeX treatment reactor operating in the temperature range from about 50° C. to 170° C., such as 60 to 80° C. Residence time can be varied from 3 min to 2 h. The treatment conditions are selected based on the desired DP of the cellulose pulp. For example, a cellulose pulp with similar DP to the starting material is associated with lower temperatures and shorter residence times while a cellulose pulp with lower DP than the starting material is associated with higher temperatures and longer residence times. Treatment conditions may also vary depending on the composition of waste textiles because these conditions need to be adapted to the special characteristics of cellulose II, usually by lowering the temperature and shortening the residence time to account for the higher accessibility and lower DP of the material.

In one embodiment the present invention targets a minimum formation of monosaccharides, oligosaccharides and low molecular weight chains with DP under about 150. Cotton that has a high DP (degree of polymerization) in the range of 10 000 can be subjected to harsher conditions in the dissolution step than shorter low crystallinity DP fibers, such as viscose fibers and/or cold alkali fibers. In a tubular reactor design for the MeX mixing step, waste cotton can advantageously be comminuted first, preferably by milling, and then subjected to harsh conditions followed by feeding viscose and other shorter DP fibers in a later stage in the reactor at milder conditions.

Cellulose solutions in the concentration range of 6-12% by weight can be prepared keeping DP equal to or above 400 and using ZnCl₂ as the MeX solvent. The process can also be controlled (MeX concentration, time, temperature) to depolymerize the cellulosic material to a desired range, for example DP 180-200.

For cotton textile waste material that comprises cellulose polymers with a cellulose DP in the range of 8000-15000, an activation stage prior to the main dissolution occurring during the mixing step of the present invention may have to be used to effectively dissolve and partly depolymerize the cotton. Such an activation stage is preferably performed by treatment of concentrated metal halide MeX in a ball mill as described herein, by steam explosion (STEX) treatment, or by hydrothermal treatment (100-200° C. for 5 min to 3 hours in the presence of an organic acid such as citric acid).

Polyester and most other synthetics and wool are not solubilized and, according to an embodiment, are separated prior to separation of amorphous cellulose as described herein.

An aqueous liquid is added thereafter, enforcing precipitation of the amorphous cellulose, which in turn is separated from the MeX solvent by filtration or centrifugation or any other suitable process for separation of solids and liquids. The aqueous liquid comprising MeX is optionally concentrated, for example, by evaporation and the condensate is recycled thereafter for reuse in the precipitation step. Preferably, more than 90% of the cellulosic components, more preferably 95%, in the waste textiles are recovered as cellulose pulp from the MeX treatment.

A more detailed description of the MeX step is disclosed below using ZnCl₂ as MeX solvent.

Control of DP (Degree of Polymerization)

Methods for controlling the DP of the cellulose pulp produced in accordance with the invention include adding an acid to be present in the MeX stage, such acids including hydrochloric acid. Such an acid may be added during an adaption step. The adaptation may thus be a separate step, for example subsequent to having recovered the cellulose pulp. It may, however, also be part of the initial heating step, dissolving the cellulose. The operating conditions of the MeX mixing and heating steps, including residence time and temperature, can also be modified to achieve a certain degree of DP change of the resulting amorphous cellulose polymers.

Control and adjustment of the DP of the cellulose can also be performed in a downstream bleaching step, which may include oxidation with oxygen and strong oxidants, such as ozone or hydrogen peroxide, under alkaline or acidic conditions, enzymatic treatment, hydrolysis (acid or alkaline catalyzed). For example, an oxidant alone or together with a metal, such as iron or manganese, may be introduced into a de-polymerization step to achieve the desired level of de-polymerization of cellulose polymers. A chlorine dioxide stage may be operated at harsher acidic conditions. The cellulose pulp material may be purified and hydrolyzed to the desired DP level by treatment with acids, such as sulfuric acid.

The DP can also be controlled and adjusted to a certain extent upstream of the MeX stage by, for example, physical/mechanical degradation (e.g. via the thermomechanical energy input of the processing equipment), such as, for example, hydrothermal and steam explosion treatments, or combinations thereof.

Thus, there is a toolbox of various methods to control and adjust the DP in to the desired range for specific end use of the dissolving pulp. According to an embodiment, the average DP of the recovered cellulose pulp is from about 200 to about 550. According to another embodiment, the average DP in the resulting pulp is from 80 to about 150.

ZnCl₂ Solutions, Hydration Levels and Amount of Antisolvent

Whether the ZnCl₂ hydrate can dissolve cellulose or only swell it greatly depends on the amount of water present. The occurrence of structural changes within the cellulose's crystal lattice is dependent on whether the cellulose has only been swollen by ZnCl₂ hydrate or has been dissolved. The swelling of cellulose with this salt hydrate has been described to affect the cellulose I crystal structure only slightly or not at all, while dissolution and subsequent regeneration of cellulose obviously yields a cellulose II crystal lattice. ZnCl₂ treatment significantly increases the fibrillation tendency of the cellulose fiber. The treatment proposed herein may thus, depending on the temperature and residence time in the heating step, affect at least a partial phase transition from cellulose I into II.

Due to their low toxicity and specific interactions with cellulose, Zn compounds are used widely in the textile industry, for example for achieving permanent crepe-like effects in textile finishing and as additives in viscose spinning.

As alluded to herein, one objective of the present invention is to at least partially dissolve the cellulose polymers present in the cellulosic waste textile substrate in the MeX mixing and heating steps. The optimum concentration to be used varies with the used type of MeX hydrate/solvent.

Zinc chloride hydrate is an acidic solvent due to both the Lewis acidity of the Zn²⁺ and the Brønsted acidity of the coordinated water, which can not only dissolve but also partially break down cellulose. It is assumed that the ionic liquid structure of the R=3 zinc chloride hydrate melt [Zn(OH₂)₆] [ZnCl₄], which is nonpolar but a strong hydrogen bond donor, may account for its ability to dissolve cellulose.

Therefore, the ionic liquid structure of the three-equivalent hydrate of zinc chloride (ZnCl₂·R H₂O, R=3, existing as [Zn(OH₂)₆] [ZnCl₄]) could explain the solubility of cellulose in this medium. Only hydrate compositions in the narrow range of 3−x<R<3+x with x≈1 effectively dissolve cellulose.

The concentration of ZnCl₂ in the feed to the mixing step of the present invention should preferably be in the range of 60 to about 78%. Concentrations below 60% ZnCl₂ are less effective due to the water molecules competing with the hydroxyl groups in cellulose to create interactions with zinc chloride. Higher concentrations only swell the cellulose. According to an embodiment, the comminuted textile material is swollen in the presence of ZnCl₂ and subsequently dissolved by diluting the ZnCl₂. At low concentrations of ZnCl₂, the reactive sites are saturated with water and can therefore not dissolve, but only swell cellulose. It has been shown that the crystal structure is preserved for treatments at 40° C. using 64 and 68 wt %

Once dissolved, the cellulose stays in solution until a water or aqueous solution is added in the precipitation step. Contrary to the established opinion in the art, it was unexpectedly found that the present invention may require a smaller amount of antisolvent to precipitate cellulose from the MeX solution when cotton fibers are used as the starting material. Especially, it was found that cellulose solution of cotton fibers becomes unstable upon addition of water well before R in ZnCl₂·R H₂O equals or exceeds 9, i.e. an addition of water to have R equal 9, i.e. nonahydrate, is not required to destabilize a solution of cotton fibers. In particular, adding water to have R=7.5 was found to be sufficient to precipitate cellulose from cotton fiber solutions, which points to the fact that solutions of waste textiles with high DP are more unstable than reported in previous art. This finding is also consistent with the fact that cotton fibers may maintain their crystallinity during MeX treatment, unless harsher conditions are utilized. This finding is opposite to what has been reported in previous art, where decrystallization of the material is usually achieved independently of the treatment conditions, see e.g. “High enhancement of the hydrolysis rate of cellulose after pretreatment with inorganic salt hydrates” by Lara-Serrano et al. (Green Chemistry, 2020, 22 (12), pp. 3860-3866, doi 10.1039/d0gc01066a).

On the contrary, man-made cellulose fiber solutions require a larger amount of aqueous solvent, e.g. water, than the R=9 hydrate to completely precipitate the cellulose. Even if precipitation starts taking place at the R=9 hydrate, a significant amount of additional antisolvent is required to completely separate the cellulose pulp from the solvent with at least 90% mass recovery. In some cases, the R=20 hydrate needs to be achieved. Without being bound by any theory, the most likely explanation for this is the exceptional ability of the cellulose pulp to absorb liquid, thanks to its lower DP, which could create local spots of concentrated MeX inside the pulp despite having a lower concentration in the free liquid.

Thus, the amount of water to be used in the precipitation step may be selected based on the particular characteristics of the starting material, in order to be able to tolerate a broad spectrum of waste textiles and reduce the requirements imposed on the feedstock of the process.

According to an embodiment, wherein crude waste textile material comprises cotton fibers, the aqueous solution is added to provide a molar ratio of H₂O:MeX not exceeding 9 in the step of precipitating the cellulose pulp. Typically, the aqueous solution is added to provide a molar ratio of H₂O:MeX of at least 7.5 in the step of precipitating cellulose pulp.

According to an embodiment, wherein crude waste textile material comprises regenerated cellulose fibers the aqueous solution is added to provide a molar ratio of H₂O:MeX exceeding 9 in the step of precipitating cellulose pulp. The aqueous solution may be added to provide a molar ratio of H₂O:MeX of 15 to 25 in the step of precipitating cellulose pulp.

The desired ZnCl₂ tetrahydrate ZnCl₂(H₂O)₄ crystallizes from aqueous solutions of zinc chloride, which greatly facilitates recovery by crystallization. Other methods to produce the desired hydrate include amminization and hydrometallurgical processing to prepare anhydrous ZnCl₂.

Removing Catalyst Poisons

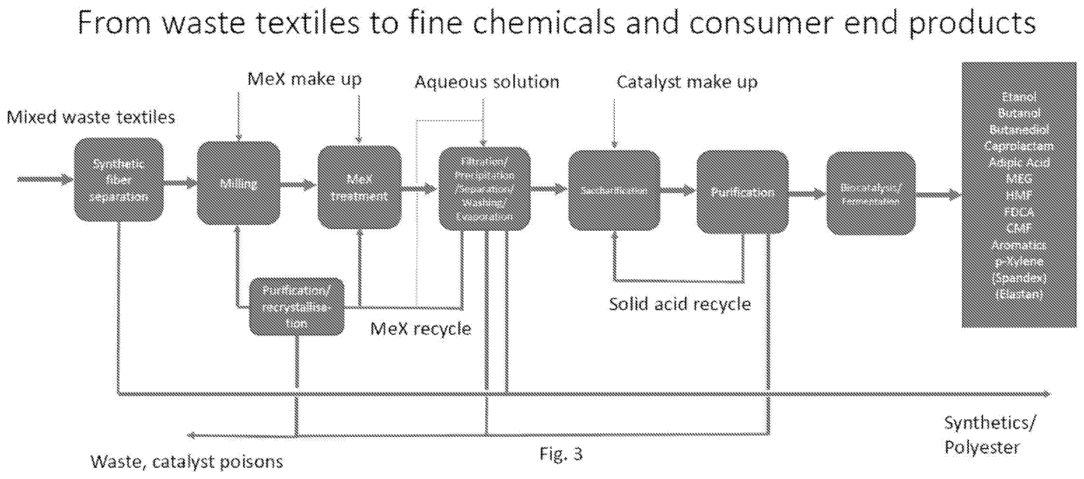

Catalyst poisons present in the crude waste textile material may, at least partly, be removed in the present process. As an example, catalyst poisons may be removed from the MeX prior to being recycled to treat new waste textiles. The metal halide MeX in the form of, for example, ZnCl₂ may for example be purified by recrystallization from hot dioxane or treatment with thionyl chloride and thereafter discharged to a metal recovery plant/melter for recycle. This purification/recrystallization is shown in FIG. 3.

Further, a continuous cellulosic MeX waste textile treatment process may comprise a minor bleed off stream to prevent non-process elements enrichment in the loop. Consequently, it may be necessary to add a make-up to restore the material balance in the chemicals recycling loop, as shown in FIG. 3.

Downstream Processing of Dissolving Pulp

Several methods have been proposed for the separation of glucose from acid and/or metal halide solutions, but they typically require a large amount of energy and solvent, which leads to the generation of large amounts of waste. For example, U.S. Pat. No. 4,452,640, disclosing a method for hydrolyzing e.g. cotton linters to glucose, states that glucose and ZnCl2 (used to hydrolyze the cotton linters) are difficult to separate and suggests using ion exclusion with an anion exchanger to separate glucose and ZnCl2 from the aqueous product solution. U.S. Pat. No. 4,018,620 employs CaCl2) as a cellulose swelling agent, and the cellulose swelling agent is separated by crystalizing CaCl2) as a hexahydrate or by adding sulfuric acid into the aqueous product solution to form calcium sulfate precipitate. Extraction with amines in the presence of a solvent has been proposed to minimize separation costs, as disclosed in EP 3 257 954 B1.

However, any residual ZnCl2 present in the cellulose pulp may be removed by washing with water and/or additional washing with a 1 wt % aqueous NaOH solution as the cellulose has been precipitated. This means that the present invention allows an easy and energy-efficient separation of the cellulose and the metal halide, avoiding the problematic sugar/acid separation.

Mixed Textile Waste (Polyester Separation from Cellulosics)

The creation of a true circular economy for cellulose-based crude waste textile material must adhere to the fact that waste textiles are often mixed and need to include an upcycling method for cotton polyester blended waste garments.

Polyesters can be composed of a great variety of polymers including Polyglycolic acid (PGA), Polylactic acid (PLA), Polycaprolactone (PCL), Polyethylene adipate (PEA), Polyhydroxyalkanoate (PHA), Polyethylene terephthalate (PET), Polybutylene terephthalate (PBT), Polytrimethylene terephthalate (PTT), Polyethylene naphthalate (PEN) or combinations thereof.

Many fabrics are blends of materials. For example, it is quite common for garments to include both cotton and polyester. These blended fabrics are difficult to recycle as the mixed textiles must be separated mechanically, i.e. cut up and torn into smaller parts. One of the disadvantages of mechanical recycling is that the quality of the fabric, in particular the cellulosic fabric, deteriorates.

The most frequently used method for recycling waste textiles comprising cellulosic fabrics as well as synthetic fabrics such as polyester, is based on mechanical separation. However, the different techniques of mechanical separation do not always allow efficient separation of the two constituents, as they are often too intimately linked in a single fabric (e.g. polycotton). Similarly, e.g. synthetic threads in cotton garments may be difficult to efficiently remove by mechanical separation.

A mechanical separation step should preferably be followed by other treatments including chemical treatments. Hard objects such as buttons and zippers should to be removed from the garment upstream of any chemical pretreatment. The garment should thereafter be cut into appropriately sized pieces.

Two chemical routes are possible:
Dissolution and total or partial selective depolymerization of cotton,
Dissolution and total or partial selective depolymerization of the polyester/synthetic.

Whatever the route taken, the dissolution must be selective, so that one or more solvents must be used, capable of dissolving one of the constituents, but inert with respect to the other constituent.

The procedure for valorizing mixed textile waste comprising polyesters in accordance with the present invention may include two steps for separation of the polyester fraction from the cellulosic stream:
a) Optional mechanical separation of polyester and other synthetics upstream of the comminuting/MeX steps of the process of the present invention by, for example, optical methods. NIR technology such as near infra-red spectroscopy can be used to identify garments made of pure cotton, polyester, acrylic, wool, polyamide, silk and man-made cellulosic (viscose, modal and lyocell) fibers as well as cotton/polyester blends.

Optionally or combined with mechanical separation, the stream of mixed textiles may be subjected to a hydrothermal treatment. The hydrothermal treatment requires a step-by-step process in which the material can be broken down into its original chemical components and cellulosic fibers, often in the presence of an aqueous alkaline catalyst (NaOH), which however complicates the process as sodium sulfate waste is formed after neutralization. Up to 30% of the cotton cellulosics may also become dissolved, which may lower the overall yield of valuable biochemicals in a downstream fermentation/bio catalysis step on sugars derived from cellulosics. The hydrothermal process is completed in a half to two hours' time frame, resulting in the decomposition of cotton fibers while the polyester fibers remain intact and can be separated.

Another alternative route for upstream separation of polyester and cellulosics is to dissolve the polyester upstream of the comminuting, e.g. milling, step of the present disclosure in a solvent that does not dissolve cellulose, and thereafter separate the dissolved polyester material. Several solvents are known for dissolving polyesters such as para-chloroanisole, nitrobenzene, acetophenone, propylene carbonate, dimethyl sulfoxide, 2,6 xylenol, quinoline, trifluoroacetic acid, ortho-chlorophenol, trichlorophenol, mixtures of trichloroethane and phenol, trichloroacetic acid and dichloromethane, trichloroacetic acid and 1,1,1 trichloroethane, trichloroacetic acid and water, trichlorophenol and phenol, 1,1,2,2 tetrachloroethane and phenol, 1,1,2 trichloro 1,2,2 trifluoroethane and 1,1,1,3,3,3 hexafluoro-isopropanol. Other known polyester solvents and solvent systems that are useful include compounds having at least one or more, commonly two or more, condensed rings in their structure such as diphenyl, diphenyl ether, naphthalene, methylnaphthalene, benzophenone, diphenylmethane, para-dichlorobenzene, acenaphthene, phenanthrene and similar compounds. Naphthalene is highly selective for polyesters in the sense that although minor amounts of nylon 66 (<0.1%) may dissolve, it will not, at up to 220° C., dissolve other common fibers including acetate, cotton, rayon, wool, nylon-6, acrylic, glass and metallic fibers. Also, certain ionic liquids dissolve polyester selectively.
b) Separation of synthetic fiber material in the filtration/precipitation/separation/washing separation steps of the present invention. The cellulosics are dissolved in the MeX solution and the synthetics in solid or fibrous form can be separated by any solid/liquid separation method such as centrifugation, filtration gravity separation, etc. An aqueous solution is then added to precipitate highly accessible cellulose for use, for example, to prepare cellulose acetate, and/or cellulose ethers, for use as a dissolving pulp for preparation of regenerated cellulosic fibers or selective saccharification to glucose. The cellulose product is separated from the MeX solution, which is recycled for treatment of new waste textiles. The recovered cellulose pulp can be bleached to increase brightness. Such bleaching is preferably performed by ozone, peroxide or chlorodioxide treatment in standalone bleaching plants or in CTMP or kraft pulp mill bleaching plants.

Integration with Pulp Mills

The possibility to integrate the present process in a sulfite mill is of special interest since the bleaching sequence typically used at these facilities may also increase the reactivity of the present cellulose; be it cellulose in the crude textile material or in the recovered cellulose pulp. Such a bleaching may consist in a pressurize oxygen treatment in the presence of Mg(OH)₂. The effluents of the bleaching sequence could be conveniently disposed of in the MgO boiler installed at sulfite mills, minimizing the waste of the process and therefore reducing the operational costs. According to an embodiment, the comminuted textile material is bleached in a sulfite mill before being mixed with the metal halide. Such an initial bleaching step will not only increase the reactivity of the cellulose, but also decrease the amount of contaminants. Thereby, purification and recycling of the metal halide used as solvent becomes easier. Further, also the recovered cellulose pulp may be bleached in a sulfite mill. Thus, the present process may comprise a bleaching step being performed in a sulfite mill. Further, integration in a sulfite mill provides for the possibility to send effluents of the present process to the waste management system of a sulfite mill.

Alternatively, the MeX treatment can be combined with alkaline treatment in a kraft mill, i.e. the precipitated cellulose pulp may be subject to alkaline treatment in a kraft mill. Alkaline treatment does typically not dissolve the cellulose, but only swells it. It may be of interest to subject the precipitated cellulose pulp to alkaline treatment in a kraft mill in embodiments wherein eventual depolymerization of the cellulose is intended. Green liquor, one of the internal streams in kraft mills, is rich in alkaline compounds (especially Na₂CO₃) so the mill could provide the necessary chemicals in the alkaline treatment at no additional cost. This would reduce the operational costs of the present invention, thanks to the reduction of the chemical purchases, and at the same time expand the portfolio of products at the kraft mill, creating a synergy between the two processes. The alkali-treated textiles can be depolymerized to glucose via acid and/or enzymatic hydrolysis, whose efficiency is greatly enhanced thanks to the swelling effect exerted during the alkaline treatment.

Examples of End Products Derived from Waste Cellulosic Fibers and Garments

Cellulose Pulp

The recovered cellulose pulp may comprise contaminants. Further, it may be discolored. According to an embodiment, the recovered cellulose pulp is thus subjected to:
one or more washing steps to remove impurities, such as metals and inorganic salts; and/or
one or more bleaching steps; preferably bleached to an ISO brightness exceeding 90 according to ISO 3688:1999.

A dissolving cellulose pulp produced in accordance with the invention is a useful starting material for various processes. As outlined in FIG. 2, it may be processed and used in various manners. Generally, it may be used in applications relying on the presence of a cellulosic polymer or, especially if the average DP in the waste textile feedstock is low, used in a process directed to the conversion to monomers, e.g. glucose, or into other fine chemicals such as CMF (Chloromethylfurfural) or adipic acid.

Thus, the provided cellulose pulp may, for example, be used in spinning new cellulosic fibers, such as in a viscose process, or a process not involving any derivatisation, such as a cold alkali spinning process. Further, dissolving pulp may be derivatized to provide dialcohol cellulose, carboxymethyl cellulose, cellulose acetate and/or cellulose ethers.

Further, the cellulose pulp, comprising purified cellulosic polymers, is useful in processes wherein monomers (e.g. glucose) are used as basis for further conversion. The purification and improved reactivity and solubility of cellulosic polymers implies that the cellulose pulp may be used in processes wherein the cellulosic polymers are hydrolyzed into monosaccharides, e.g. glucose using solid acids or enzymes as catalysts, where the resulting glucose solution may be converted in to other chemicals.

Various options and uses of the recovered cellulose pulp are outlined and described below. Different applications of the cellulose pulp provided from the waste textile material may require different degrees of polymerization (DP). The DP of the resulting cellulose pulp may be affected by the DP of the cellulose fibers in the waste textile material as well as processing conditions.

For various applications of the cellulose pulp, the degree of polymerization (DP) needs to be controlled by changing the conditions during treatment with a molten salt (time, temperature, concentrations, and optional addition of acid such as sulfuric or hydrochloric acid). Table 2 presents the typical desired parameters, such as the DPₙ, the number degree of polymerization typically referred to as DP. DP_{w} is the weight degree of polymerization of the dissolving pulp to produce various end-products disclosed herein.

TABLE 2
Degree of polymerization required in dissolving
pulp to produce different end-products

| | Viscose | Ether | Viscose | Acetate | Acetate |
|---|---|---|---|---|---|
| Raw material | Hardwood | Softwood | Hardwood | Softwood | Cotton |
| Cooking | Sulfite | Sulfite | PHK | PHK | Linters |
| process | | | | | |
| DP_{W} | 1800 | 4750 | 1400 | 2100 | 1250 |
| DPₙ | 280 | 450 | 460 | 650 | 700 |

Glucose Solution

In cases where the average DP of the waste textiles hinders the valorization of the cellulose pulp as a polymer, the pulp can be depolymerized to monomeric sugars and these can be used as the basis for further conversion into valuable products. The increased purity and reactivity of the pulp, resulting from the dissolution/regeneration described in the present invention, facilitates the saccharification of the material into monosaccharides with solid acids or enzymes, ensuring a high yield of monosaccharides in the operation.

The resulting glucose solution may be converted into a myriad of chemicals, as glucose is the preferred feedstock in many chemical and biochemical industrial processes. For example, adipic acid is one of the most commercially important aliphatic dicarboxylic acids. It is produced on a large scale, primarily to supply the polyamide 6.6 production chain. Other applications include the manufacture of coatings, synthetic lubricants, fibers, plastics, plasticizers, and polyurethane resins.

Adipic acid can be produced from glucose over sugar derived from cotton and viscose waste via a two-step catalytic process over glutaric acid or by microbial processes.

Several other platform organic chemicals such as ethanol, butanediol and caprolactam (caprolactone over HMF) can be produced from glucose by well-known catalytic or microbial processes, including fermentation.

Another example would be the production of furan dicarboxylic acid (FDCA), which can be used to manufacture a biobased substitute for polyester known as PEF. Recently, the company Stora Enso commissioned a pilot plant in Belgium where FDCA is produced from glucose, which highlights the industrial relevance of this application.

Glucose can also be fermented into 1,4-butanediol, which can be used as a precursor in the production of spandex/lycra, and caprolactam, which can be used as a precursor in the production of nylon 6. Thus, even in cases where the cellulose needs to be depolymerized, it is possible to keep the value of the material within the textile sector by recycling cellulosic fibers into synthetic fibers.

HMF and CMF

It is generally accepted that three steps are required for the conversion of cellulose to 5-(hydroxy¬methyl)¬furfural (5-HMF):
(1) hydrolysis of cellulose to glucose;
(2) isomerization of glucose to fructose;
(3) dehydration of fructose to 5-HMF. 5-HMF can be produced in high yield in a single step starting from MCC (microcrystalline cellulose) solution in an acidic ZnCl₂ aqueous solution. A solvent such as MIBK can be used for dissolving HMF and thereby facilitate separation of catalysts. The effect of reaction conditions on conversion was explored by varying the concentration of ZnCl₂, acid concentration, reaction time and temperature. It is of high importance to control both the concentration of ZnCl₂ and pH to prevent undesired formation of humins, formic and levulinic acid by-products. In accordance with the present invention, on the other hand the reaction conditions including concentration of MeX (such as ZnCl₂), acid concentration, reaction time and temperature must be selected to prevent formation of glucose.

In one embodiment of the present invention, a metal halide (such as ZnCl₂) and optionally an acid catalyst are used to dissolve a crude waste textile material, comprising cotton viscose and other cellulosic fiber wastes, followed by separation of the cellulose from the liquid metal halide by precipitating the cellulose pulp from the solution of cellulose and MeX. The cellulose pulp is thereafter saccharified and processed selectively to HMF by solid acids optionally supported by a homogeneous acid catalyst such as sulfuric acid, hydrochloric acid (gaseous or liquid) or sulphonic acid.

HMF can be oxidized to 2,5-furandicarboxylic acid (FDCA) with well-known methods, which in turn can be used as a renewable substitute for the petroleum-based terephthalic acid in polymer production or for bioplastic production.

5-Chloromethylfurfural (CMF) is an organic compound with the formula C₄H₂O(CH₂Cl)CHO. It consists of a furan substituted at the 2- and 5-positions with formyl (CHO) and chloromethyl (CH₂Cl) groups. CMF is obtained by dehydration of fructose and other cellulose derivatives using hydrochloric acid and/or salts. It is a colorless liquid. It can be reduced to give 5-methylfurfural and can be hydrolyzed to give hydroxymethylfurfural. CMF can be further transformed into para-xylene, terephthalic acid (PTA), used to provide polyethylene terephthalate (PET), as well as numerous commodity and specialty chemicals through its derivatives, including furan dicarboxylic acid (FDCA).

CMF preparation from biomass and cellulose and its upgrade to valuable organic chemicals is well known in the art, for example in “Dramatic advances in the saccharide to 5-(chloromethyl)furfural conversion reaction” by Mascal et al. (ChemSusChem, 2009, 2 (9), pp. 859-861, doi 10.1002/cssc.200900136). In U.S. Pat. No. 4,424,390, a process is described for producing 5-halomethylfurfural, which comprises carrying out an acid-decomposition of saccharide in a water/organic solvent/magnesium metal halide system, in the presence or absence of a surface-active agent as a catalyst.

As far as is known to the applicant, the dissolution of crude waste cellulosic textiles by metal halides, forming regenerated cellulose pulp followed by saccharification and biocatalytic production of CMF, is not described in the prior art. The obtained CMF can then be further converted into e.g. PE, PTA or FDCA as shown in FIG. 2 using processes known in the art.

CMF is a carbohydrate-derived platform molecule that is gaining attraction as a more practical alternative to 5-(hydroxymethyl)furfural (HMF). The main advantage of CMF over HMF is that it can be produced in high yield under mild conditions directly from raw biomass because its hydrophobicity markedly facilitates isolation. CMF is also a precursor to levulinic acid (LA), another versatile biobased intermediate. Examples of commercial markets that can be unlocked by synthetic manipulation of CMF are divided into two derivative pathways, furanic and levulinic, which are distributed over three product family trees: renewable monomers, fuels, and specialty chemicals. Selected examples of CMF- and LA-based routes to these products are presented.

CMF can be prepared from carbohydrates by treatment with metal chloride/concentrated hydrochloric acid solution, and organic solvent into a reactor, and stirring and reacting at a temperature of 80-180° C. for 0.5-40 h. After the reaction, extraction, separation, and purification are performed to obtain the product CMF.

DMF and p-Xylene

Catalytic in-situ hydrogenation of 5-hydroxymethylfurfural (HMF) to 2,5-dimethylfuran (DMF) has received great interest in recent years. Different reaction parameters such as catalysts, reaction temperature, time, pressure, solvents and catalyst amount have been optimized to achieve the highest conversion of HMF and selectivity of the products. High conversion of HMF (98%) and selectivity of DMF (97%) can be achieved with an Ru/ZSM-5 catalyst at 180° C. in an ethanol solvent system for 3 h in 250 psi H2. DMF can be used as a biofuel component or be further upgraded to para xylene.

p-Xylene is a major commodity chemical used to produce polyethylene terephthalate, a polymer with applications in polyester fibers, films, and bottles. The Diels-Alder cycloaddition of 2,5-dimethylfuran (prepared from, for example, HMF as per above) and ethylene, and the subsequent dehydration of the cycloadduct intermediate, is an attractive reaction pathway to produce renewable p-xylene from biomass feedstocks such as cellulose from waste cellulosic textiles. P-containing zeolite Beta is a selective catalyst for this reaction with p-xylene yield in the range of 97%.

Dialcohol Cellulose

Periodate oxidation is a unique reaction in that it selectively causes cleavage of the C2-C3 bond in the glucopyranose ring when used in cellulose. The resulting product, i.e., 2,3-dialdehyde cellulose, has an open-ring structure at the C2-C3 position, where the OH group is converted to the dialdehyde group. A long time is needed for the solid-liquid reaction. One of the easiest alternatives to shorten the reaction time is to use a solution reaction instead of the conventional solid-liquid phase reaction. However, such a reaction is difficult to achieve in the case of cellulose because of the lack of suitable aqueous solvents for cellulose, in comparison with the wide variety of non-aqueous organic solvents. One possibility is to perform the periodate oxidation in a molten salt solvent (solvent herein) and then produce the dialdehyde cellulose. Alternatively, it can be produced by a standard procedure well-known in the art.

Dewatering of the dialcohol cellulose can be performed in several ways, including liquid-liquid extraction in an organic solvent such as an alcohol.

Cellulose Acetate

Cellulose acetate refers to any acetate ester of cellulose, usually cellulose diacetate. It was first prepared in 1865. Since it is a bioplastic, cellulose acetate is used as a film base in photography, as a component in some coatings, and as a frame material for eyeglasses. It is also used as a synthetic fiber in the manufacture of cigarette filters and playing cards. In photographic film, cellulose acetate film replaced nitrate film in the 1950s, being far less flammable and cheaper to produce.

Cellulose acetate fiber, one of the earliest synthetic fibers, is based on cotton or tree pulp cellulose (“biopolymers”). These “cellulosic fibers” have been replaced in many applications by cheaper petro-based fibers (nylon and polyester) in recent decades.

Cellulose acetate shares many similarities with rayon, and was formerly considered as the same textile. However, cellulose acetate differs from viscose rayon in the employment of acetic acid in production and therefore the two fabrics are now required to be listed distinctly on garment labels. Moreover, viscose rayon is heat-resistant while cellulose acetate is prone to melting and, because of this, cellulose acetate must be laundered with care either by hand-washing or dry cleaning.

The breathable nature of cellulose acetate fabric makes it suitable for its use as lining. Cellulose acetate fabric is used frequently in wedding gowns and other bridal attire. Its lustrous sheen and smooth, satiny texture make it a good alternative to silk.

The cellulose pulp produced in accordance with this invention is particularly suitable for production of cellulose acetate. However, no effective procedure for the direct production of cellulose acetate has been found, as attempts to produce a partial esterification of cellulose result only in a mixture of non-acetylated and fully acetylated cellulose. For this reason, a two-step synthesis is applied: first, cellulose is always completely converted to cellulose triacetate and then, through hydrolysis, into cellulose acetates with low degree of esterification.

According to an embodiment, cellulose pulp obtained in the process according to the present disclosure is mixed with glacial acetic acid, acetic anhydride, and a catalyst in accordance with the general procedure for production of cellulose acetate fabrics. The resulting mixture is aged for 20 hours, during which partial hydrolysis occurs and acid resin precipitates as flakes. These are dissolved in acetone and the solution is purified by filtering. The solution is extruded by spinning in a column of warm air and the solvent is recovered. Filaments are stretched and wound onto beams, cones, or bobbins ready for use. Filaments are finally spun into fiber.

In one preferred embodiment, the acylation of cellulose is performed under homogeneous conditions using a cellulose solvent wherein the substantially amorphous cellulose produced in accordance with the present invention is dissolved. Such solvent may be an ionic liquid, for example a molten salt solvent. Such ionic liquid solvents are well known to the skilled person. Particularly advantageous solvents are molten salt solvents that comprises zinc, for example ZnCl₂ or ZnBr₂ (as described further hereinabove).

In one embodiment, acylation reagents, for example acetic acid and/or acetic acid anhydride, are added to the solution of cellulose and MeX to form cellulose acetates to be precipitated. Cellulose acetate in the desired degree of substitution (DS) ranging from 2-3 can be obtained depending on which conditions are selected in the MeX stage (temperature, addition of acid etc.). Such cellulose acetate can advantageously be used for manufacturing of textile fibers, for example with dry spinning methods. With such unit operations waste cellulosic textiles are turned into new recyclable cellulose acetate textile fibers.

Cellulose Ethers Dissolving pulp from wood is a kind of highly purified chemically refined material. It has high α-cellulose content (>90.0%), and low hemicellulose (<4.0%), lignin and other impurities (<0.5%) content. It is the starting material of multiple chemical products and it is used in the preparation of multiple cellulose products, such as qualitative and quantitative filter paper, various cellulose ethers, cellulose esters, etc.

However, the cellulose pulp produced in accordance with the present invention could have even higher purity than dissolving pulp derived from wood, due to the absence of hemicellulose and lignin in cellulose-based waste textiles, and therefore it could provide a better substrate to produce chemicals, such as cellulose ethers and cellulose esters.

Another specific feature of the cellulose pulp produced in accordance with the present invention that makes it particularly suitable for manufacturing of cellulose ethers is the low crystallinity of the pulp. The crystallinity index of the pulp is measured by X-ray diffraction methods. It is preferably lower than about 60, more preferably lower than about 50, and most preferably lower than about 30 (highly amorphous).

Carboxymethyl cellulose (CMC) is the major cellulose ether consumed worldwide, accounting for half of the total consumption in 2018. The market for CMC is divided into technical (crude), semi-purified, and high-purity grades. The largest end-uses are detergents (which utilize technical CMC), oil field applications, and food additives. It is synthesized by the alkali-catalyzed reaction of cellulose with chloroacetic acid. The polar (organic acid) carboxyl groups render the cellulose soluble and chemically reactive.

Methylcellulose and its derivatives, such as hydroxypropyl methylcellulose (HPMC), represented a third of the total consumption of cellulose ethers in 2018, while hydroxyethyl cellulose, its derivatives and other cellulose ethers accounted for the remainder. The demand for these cellulose ethers is driven by building/construction end-uses (including surface coatings) and food/pharma/personal care applications.

Ethyl hydroxyethyl cellulose is a cellulose-based product in which ethyl and hydroxyethyl groups are attached to the anhydroglucose units by ether linkages. Ethyl hydroxyethyl cellulose is prepared from cellulose by treatment with alkali, ethylene oxide and ethyl chloride. The final product may be specified further by the viscosity of its aqueous solutions.

Methyl ethyl hydroxyethyl cellulose ethers can be prepared according to conventional methods that are known to those of ordinary skill in the art. For example, alkali cellulose (activated cellulose) may be prepared in one or several steps by mercerizing cellulose with alkali, in which the alkali cellulose is further reacted in one or several steps with appropriate amounts of ethylene oxide, methyl chloride and ethyl chloride in the presence of an organic reaction medium, for instance, ethyl chloride, acetone, alkyl-blocked mono or poly ethylene glycols, isopropanol, tert-butanol, dimethoxyethane or mixtures thereof at a temperature in the range of about 50 to about 120° C.

Nanocellulose

Cellulose nanocrystals have been gaining attraction in advanced applications, such as biomedical products and hydrogels, in the past years. In fact, it has been shown that nanocellulose can be produced from cotton waste, and even viscose waste, by means of acid hydrolysis. For example, such processes are described in “Obtainment and characterization of nanocellulose from an unwoven industrial textile cotton waste: Effect of acid hydrolysis conditions” by Maciel et al. (International Journal of Biological Macromolecules, 2019, 126 (1), pp. 496-506, doi 10.1016/j.ijbiomac.2018.12.202) and in “Recycling of viscose yarn waste through one-step extraction of nanocellulose” by Prado et al. (International Journal of Biological Macromolecules, 2019, 136, pp. 729-737, doi 10.1016/j.ijbiomac.2019.06.124).

However, as far as is known to the applicant, there is no description in the prior art that mentions production of nanocellulose from post-consumer waste textiles nor the utilization of ZnCl₂ instead of acids in the hydrolysis to produce the nanocrystals. Thus, in one embodiment, the MeX treatment conditions are selected to maintain a high crystallinity in the cellulose pulp and obtain cellulose crystals with an aspect ratio in the nano scale.

According to an embodiment, at least 50 wt %. such as at least 75 or 90 wt %, of the cellulose in comminuted textile material is rich in cellulose type II. Thus, the waste textile material may comprise at least 50 wt %. such as at least 75 or 90 wt %, viscose fibers, lyocell fibers, and/or fibers cold alkali type textile fibers. Further, waste textile material may comprise less than 10 wt % cotton fibers, such as less than 5 wt % cotton fibers. In such an embodiment, the process may comprise a step of deriving cellulose rich in cellulose type II, such as nanocellulose and/or microcrystalline cellulose (MCC), from solvent once the cellulose pulp has been precipitated. Further, in such an embodiment, the process may comprise a step of deriving cellulose rich in cellulose type II, such as nanocellulose and/or microcrystalline cellulose (MCC) from an aqueous solution used to wash the precipitated pulp. The step of deriving cellulose rich in cellulose type II may comprise unit operations, such as evaporation, filtration (e.g. membrane filtration), and/or extraction. According to such an embodiment, cellulose type II present in the waste textile material may be efficiently recycled, as any cellulose type II remaining in the solvent is not discarded but recycled as well. Various use of the precipitated pulp is described herein above. Further, an overview of possible uses is given in FIG. 2.

Fiber Spinning

An alternative way of using the dissolved comminuted textile material is to spin the solution of cellulose and MeX directly into textile fibers, i.e. rather than precipitating the cellulose to provide cellulose pulp to subsequently be used in fiber spinning, the solution of cellulose and MeX may be extruded into an aqueous solution to precipitate cellulose fibers. The MeX will remain dissolved and may then easily be separated from the precipitated fibers.

### Exemplary Embodiment

In order to provide an overview on how versatile the present process is, a process scheme of an exemplary embodiment is given in FIG. 3. In short, mixed crude waste textiles comprising waste cotton, viscose, and polyester garments are freed from buttons, zippers, and other non-textile solids and thereafter shredded into smaller 1 cm×1 cm pieces. The shredded textile material is fed to a plant for separation of polyester, wherein polyester is dissolved into a solvent and separated from the solid cellulosics that are not soluble in the solvent. The separated cellulosics are charged to a ball milling unit where cotton and viscose are comminuted into fine particles. The comminuted textile material is directly charged to a mixer wherein a MeX (ZnCl₂) solvent is blended into the comminuted textile material. The mixture or slurry of comminuted textile material and MeX solvent is charged to a tubular reactor and treated at a temperature and a time sufficient to rupture the structure of cellulose and dissolve the waste textile material. The material is discharged from the reactor and solids (e.g. remaining polyester) are separated by filtration.

An aqueous liquid/solution is charged to the MeX/cellulose liquid mixture and substantially reactive pure cellulose is precipitated. MeX is purified, recrystallized, and recycled to treat new textile material in the tubular reactor.

In the embodiment shown, the substantially reactive cellulose is further charged to a saccharification plant wherein monosaccharides, primarily glucose, are produced in very high overall yield (over 80% by weight) by acidic hydrolysis using a heterogeneous solid catalyst that is recycled by simple filtration of the sugar solution. The sugar solution produced can be used for synthesis of a wide range of fine organic chemicals, such as Ethanol, Butanol, Butanediol, MEG, HMF, FDCA, CMF, etc., with processes well-known in the art, as indicated in FIG. 3.

### EXPERIMENTAL

Materials Used in the Experiments

All the materials used in the experiments described in Section 6 were actual post-consumer waste textiles that had been deemed unsuitable for reuse or resell. The waste textiles were collected and sorted by Wargon Innovation, who also removed zippers, labels and other non-process elements from the materials. The materials selected for the experiments were classified either as 100% cotton or 100% viscose, depending on the type of experiment, and they were white to avoid the need for bleaching. After sorting and collection, the textiles were cut into pieces of approximately 1 cm×1 cm prior to the experiments.

The cotton waste textiles used in the experiments had an average DP of approximately 2200 (corresponding to an IV of 620 mg/L) and exhibited a cellulose I structure with very high crystallinity (crystallinity index of 88%). The viscose waste textiles had a lower average DP of approximately 180 (corresponding to an IV of 150 mg/L) and exhibited primarily a cellulose II structure with lower crystallinity.

DP Determination Through Viscosity Measurements

The average DP of cellulose fibers can be estimated through IV measurements in different solvents. According to ISO-5351:2010, the IV of dissolving pulp is determined by dissolving the material in 0.5 M cupriethylenediamine solution and measuring the limiting viscosity in a capillary-tube viscometer.

There are several correlations to estimate the average DP from the IV of the material. In the present invention, it was decided to apply Equation 1 within a DP range of 700<DP<5000 and Equation 3 outside this range. Both equations are usually required to cover the wide spectrum present in cellulosic waste textiles, from cotton fibers with very high DP to cold alkali fibers with very low DP.

DP^{0.9}=1.65·η (Evans & Wallins, 1989) (1)

DP^{0.905}=0.75·η (Sihtola et al., 1963) (2)

The Evans & Wallins equation (Equation 1) is in truth an update of the Sihtola et al. equation (Equation 2) which, according to the authors, provides more accurate estimates of the average DP and therefore it was decided to use the Evans & Wallins equation in the experiments below.

Measurement of XRD Crystallinity

Cellulose was analyzed with X-ray diffraction (XRD) at an external laboratory to identify its morphological structure and crystallinity. The morphology of the crystals was determined by the positions of the peaks, which are on 2θ=22.6° for type I (the [200] reflection) and on 2θ=22° for type II (the [110] and [020] reflections). The crystallinity index (CI) was determined according to Segal's formula where CI is calculated as the height ratio between the intensity of the crystallinity peak (I₀₀₂−I_{AM}) and total intensity (I₀₀₂) after subtraction of the background signal measured without cellulose (the so-called peak height method).

However, it is important to mention the limitations of CI in describing the crystallinity of cellulose since this value only takes into account certain crystalline planes and therefore CI values of cellulose samples with different morphology should not be compared with each other. For this reason, changes in crystallinity are also assessed through qualitative comparison with the diffractogram of the starting material, in order to evaluate whether the cellulose pulp maintained a similar crystal structure through the treatment or not.

Cellulose Hydrolysis Yield

The enzymatic hydrolysis yield was based on the amount of glucose available in the cellulose pulp, which corresponds to 1.11 times the amount of cellulose contained in the material, due to the addition of water during the hydrolysis reaction. The yield of CMF was calculated on the same basis and the concentration of the product present in the organic phase after the reaction had been completed.

### Experiment A1: Control of DP in Dissolving Pulp Derived from Cotton Waste

The cellulose dissolution trial according to Experiment A was started by mixing 250 g of cotton waste textiles with 10 kg of 65% ZnCl₂ aqueous solution. The mixture was stirred at 65° C. for 20 min, followed by further agitation at 70° C. for 30 min. The resulting cellulose solution was diluted with 3 kg of water (0.3 g water/g solvent, corresponding to the R=7.5 hydrate) to precipitate the cellulose pulp, which was washed with abundant water to remove any residual ZnCl₂ from it. The cellulose pulp had an average DP of approximately 2160, which corresponds to almost 96% of the average DP of the starting material.

### Experiment A2: Control of DP in Dissolving Pulp Derived from Cotton Waste

Experiment A2 was performed in the same manner as Experiment A1, except for the fact that the mixture was stirred at 80° C. for 60 min, instead of 65° C. In this case, the resulting cellulose pulp had an average DP of approximately 855, which corresponds to 40% of the average DP of the starting material. Thus, this example showcases the possibility to control the average DP of cellulose derived from cotton post-consumer waste textiles by simply changing the conditions, i.e. the temperature, of the ZnCl₂ treatment.

### Experiment B1: Control of DP in Dissolving Pulp Derived from Viscose Waste

The first cellulose dissolution trial in Experiment B was started by mixing 250 g of viscose waste textiles with 10 kg of 65% ZnCl₂ aqueous solution. The mixture was stirred at 65° C. for 20 min and the resulting cellulose solution was mixed with 13 kg of water (1.3 g water/g solvent, corresponding to the R=19 hydrate) to precipitate the cellulose pulp, which was washed with abundant water to remove any residual ZnCl₂ from it. The resulting cellulose pulp had an average DP of approximately 165, which corresponds to almost 92% of the average DP of the starting material.

### Experiment B2: Control of DP in Dissolving Pulp Derived from Viscose Waste

Experiment B2 was performed in the same manner as Experiment B1, except for the fact that the mixture was stirred at 70° C. for 50 min, instead of 20 min at 65° C. In this case, the resulting cellulose pulp had an average DP of approximately 95, which corresponds to 53% of the average DP of the starting material.

Thus, Experiment B showcases two important aspects of the present invention: i) average DP of the cellulose pulp can also be controlled when regenerated cellulose fibers are used as starting material; and ii) the conditions of the ZnCl₂ can be modified so that the present invention can be applied to any type of cellulosic waste fibers, regardless of their average DP, crystallinity and crystal morphology.

### Experiment C1 and C2: Control of Cellulose Microstructure in Dissolving Pulp Derived from Cotton Waste

The first cellulose dissolution trial in Experiment C started by mixing 250 mg of cotton waste textiles with 10 g of 65% ZnCl₂ aqueous solution. The mixture was stirred at 70° C. for 60 min. The resulting cellulose solution was mixed with 3 g of water to precipitate the cellulose pulp, which was washed with abundant water to remove any residual ZnCl₂ from it.

The second dissolution trial in Experiment C was performed in the same manner as the first one, except for the fact that the mixture was stirred at 80° C. for 60 min, instead of 70° C.

The cellulose pulp resulting from the first trial Experiment C1 exhibited the typical XRD peaks corresponding to cellulose I and a high crystallinity (CI of 83%), whereas the cellulose pulp resulting from the second trial Experiment C2 exhibited the typical XRD peaks corresponding to cellulose I as well, but it contained a significant portion of amorphous material (CI of 69%). This experiment showcases that ZnCl₂ treatment can alter the microstructure of the cellulose pulp differently depending on the treatment conditions and therefore the present invention would also allow to control the crystal type and crystallinity of the pulp as well.

### Experiment D: ZnCl₂ Treatment to Enhance Saccharification of Waste Textiles

Cellulose pulp was prepared by mixing 250 g of cotton waste textiles with 10 kg of 65% ZnCl₂ aqueous solution. The mixture was stirred at 80° C. for 60 min and the resulting cellulose solution was mixed with 3 kg of water to precipitate the cellulose pulp, which was washed with abundant water to remove any residual ZnCl2 from it.

Enzymatic hydrolysis was performed on the resulting cellulose pulp as well as the starting cotton waste textiles, and non-woven cotton material (medical cotton), respectively, which were used as a control (negative and positive, respectively). These experiments were performed at 50° C., pH=6 and for 96 h in 50 mL Falcon tubes with a working mass of 20 g. A solids loading of 5% and Cellic CTec 2 (Novozymes, Denmark) enzyme cocktail at a loading of 0.15 g enzymes/g solids were used. The Falcon tubes were placed in a combi-H12 hybridization incubator (FinePCR, South Korea), which maintained the temperature and mixing during the experiments.

A glucose yield of 85.6% (ZnCl₂ treatment), 30.3% (cotton waste textiles) and 65.0% (medical cotton) was obtained from the cellulose pulp, starting cotton waste textiles and non-woven cotton material, respectively, based on the glucose concentration present in the liquid after enzymatic hydrolysis.

It could this be concluded that the cellulose pulp obtained in accordance to the present invention has a much higher reactivity than the starting material (negative control). The enhancement was so marked that the pulp obtained by dissolving cotton waste textiles even hydrolyzed better than material readily available to the enzymes, such as non-woven cotton (positive control).

### Experiment E: Production of CMF from Dissolving Pulp Derived from Cotton Waste

Cellulose pulp was prepared by mixing 30.45 g of cotton waste textiles with 580 g of 65% ZnCl₂ aqueous solution. The mixture was stirred at 70° C. for 30 min and the resulting cellulose solution was mixed with 150 mL of water to precipitate the cellulose pulp, which was washed with abundant water to remove any residual ZnCl₂ from it.

The CMF experiment starting by placing approximately 1 g of the (wet) cellulose pulp on a glass filter and rinsing it with 12 M hydrochloric acid. The dissolving pulp was transferred to a 10 mL head space vial and 12 M hydrochloric acid was added to obtain a total volume of 2 mL. Then, 4 mL of 1,2-dichloroethane (corresponding to an aqueous/organic phase ratio of 1:2) were added to obtain a cellulose pulp/total liquid ration of 1:20. The vial was capped and heated at 120° C. in a heating block for 30 min. Then the vial was placed on an ice bath, the cap was removed, the contents were filtered on a glass filter and the phases were analyzed by GC-cFID. The chromatographic analysis showed that the amount of CMF present in the organic phase corresponded to a yield of 68%.

### Experiment F: Production of CMF from Dissolving Pulp Derived from Viscose Waste

The same procedure as in experiment E was applied to produce CMF from viscose waste textiles, except for the fact that the CFM reaction was performed at 100° C. for 45 min. A CMF yield of 54% was obtained from the e cellulose pulp.

1. A process for valorization of a crude waste textile material comprising cotton fibers, viscose fibers and/or other cellulosic textile fibers into cellulose pulp, said process comprising the steps of:
providing a comminuted textile material comprising cotton fibers, viscose fibers and/or other cellulosic fibers;
mixing the comminuted textile material with at least one solvent in the form of an aqueous metal halide (MeX), comprising MeX in a concentration in the range of 60 to 80 wt %, or in the form of a metal halide hydrate having the formula MeX·n(H₂O), wherein the metal Me is Li⁺, Zn²⁺, Mg²⁺, or Ca²⁺, wherein the halide X is Cl⁻ (chlorine) or Br⁻ (bromine), and wherein the integer “n” is 2 to 6, such as 3 or 4;
heating the mixture of the comminuted textile material and the metal halide to dissolve, at least partly, the comminuted textile material to provide a solution of cellulose and MeX; and
precipitating cellulose pulp from the solution of cellulose and MeX, preferably by adding an aqueous solution to the solution of cellulose and MeX to precipitate cellulose pulp;
recovering cellulose pulp.

2. The process according to claim 1, wherein said MeX is ZnCl₂·3(H₂O), ZnCl₂·4(H₂O), ZnBr₂·3(H₂O), ZnBr₂·4(H₂O) or a mixture of two or more of these; preferably MeX being ZnCl₂·4(H₂O).

3. The process according to claim 2, wherein said MeX is ZnCl₂·4(H₂O); preferably the process further comprising the step of recovering purified MeX as ZnCl₂·4(H₂O) and recycling it to the step of mixing the comminuted textile material with aqueous MeX.

4. The process according to claim 1, wherein the solvent is an aqueous metal halide (MeX) comprising MeX in a concentration in the range of 65 to 75 wt %; preferably MeX being ZnCl₂, ZnBr₂, or a mixture thereof.

5. The process according to claim 1, wherein the process further comprises restoring the solvent and recycling at least a portion of said restored solvent comprising MeX to said step of mixing the comminuted textile material with MeX.

6. The process according to claim 5, wherein said step of restoring, e.g. concentrating, the solvent, comprises purifying MeX by at least one of evaporation, crystallization, and extraction; preferably wherein MeX is ZnCl₂ restored to and recycled as ZnCl₂·4(H₂O).

7. The process according to claim 6, wherein the step of restoring the MeX solvent comprises removing heavy metals, such as by washing solid MeX with water; preferably wherein the resulting aqueous solution comprising heavy metals is depleted of heavy metals, e.g. by membrane filtration, evaporation or precipitation, preferably at least part of the aqueous solution depleted of heavy metals being used to form part of the aqueous solution in the step of precipitating cellulose pulp.

8. The process according to claim 1, wherein the process further comprises deriving cellulose of type II, such as nanocellulose and/or microcrystalline cellulose (MCC), from the MeX solvent; preferably the cellulose of type II being recovered before restoring the solvent; and/or
wherein the process further comprises deriving cellulose of type II, such as nanocellulose and/or microcrystalline cellulose (MCC), from an aqueous solution used to wash the precipitated pulp.

9. The process according to claim 1, wherein the step of heating the mixture of the comminuted textile material and the metal halide is performed at a temperature in the range of 50° C. to 170° C., preferably in a temperature range of 60° C. to 80° C.; and/or wherein the mixture of the comminuted textile material and the metal halide is heated during a time range of 3 minutes to 4 hours.

10. The process according to claim 1, wherein crude waste textile material comprises cotton fibers, and wherein the step of heating the mixture of the comminuted textile material and the metal halide is performed at a temperature in the range of 70° C. to 100° C., preferably in the range of 75° C. to 85° C.; and/or wherein the mixture of the comminuted textile material and the metal halide is heated during a time range of 1 to 2 hours.

11. The process according to claim 10, wherein the aqueous solution is added to provide a molar ratio of H₂O:MeX not exceeding 9 in the step of precipitating the cellulose pulp.

12. The process according to claim 1, wherein crude waste textile material comprises regenerated cellulose fibers, and wherein the step of heating the mixture of the comminuted textile material and the metal halide is performed at a temperature in the range of 50° C. to 80° C., preferably in the range of 60° C. to 70° C.; and/or wherein the mixture of the comminuted textile material and the metal halide is heated during a time range of 15 to 45 minutes.

13. The process according to claim 12, wherein the aqueous solution is added to provide a molar ratio of H₂O:MeX exceeding 9 in the step of precipitating cellulose pulp.

14. The process according to claim 1, wherein the crude waste textile material is comminuted by at least one of shredding, grinding and milling, or a combination thereof, to provide a comminuted textile material.

15. The process according to claim 14, wherein the crude waste textile is comminuted by milling; optionally wherein MeX is added to be present in the milling step.

16. The process according to claim 1, wherein any fraction of polyester present in the crude waste textile material is, at least partly, separated prior to adding the aqueous solution to precipitate the cellulose pulp; preferably the polyester fraction being separated by;
dissolving the polyester in a solvent and separating the solid cellulosic textile fibers by a solid liquid separation process prior to mixing the cellulosic fibers with MeX; and/or
separating a solid polyester fraction from the solution of cellulose and MeX by a solid liquid separation process after having dissolved the textile fibers to provide a solution of cellulose and MeX, but before precipitation pulp.

17. The process according to claim 1, wherein the crude waste textile material is subject to a sorting step prior to mixing the comminuted textile material with at least one solvent in the form of an aqueous metal halide (MeX), said sorting step serving to:
remove metal pieces, such as buttons, rivets, and/or zippers;
remove plastic pieces, such as buttons and/or zippers;
enrich a given type of textile fibers, such as viscose fibers or cotton fibers.

18. The process according to claim 1, wherein the average degree of polymerization (DP) of cellulose in the cellulosic textile fibers in the crude waste textile material to be mixed with the solvent is lower than 700; preferably wherein the crude waste textile material comprises viscose fiber; more preferably wherein the crude waste textile material comprises at least 50 wt % viscose fiber, such as at least 75 wt % viscose fiber.

19. The process according to claim 18, wherein the average degree of polymerization (DP) of cellulose in the cellulosic textile fibers in the crude waste textile material to be mixed with the solvent is lower than 500; and/or wherein the crude waste textile material comprises at least 50 wt %, such as at least 75 wt %, cellulosic textile fibers in which the cellulose has an average degree of polymerization (DP) of less than 400.

20. The process according to claim 1, wherein the process comprises a step of reducing the average degree of polymerization (DP) of the resulting cellulose pulp, such as an adaptation step in which the cellulose pulp is treated with an acid, such as sulfuric acid, and optionally heated.

21. The process according to claim 1, wherein the average degree of polymerization (DP) of the resulting cellulose pulp is 80 to 100% of the average degree of polymerization (DP) of the cellulose in the cellulosic textile fibers in the crude waste textile material; particularly wherein the average degree of polymerization (DP) of the cellulose in the cellulosic textile fibers in the crude waste textile material is 700 or lower; and/or wherein the crude waste textile material comprises viscose rayon fibers and/or cold alkali type textile fibers.

22. The process according to claim 1, wherein the average degree of polymerization (DP) of the resulting cellulose pulp is 80 to 200, or 400 to 600.

23. The process according to claim 1, wherein the cellulose pulp is precipitated by;
adding an aqueous solution comprising an alcohol, e.g. methanol, ethanol or propanol, a ketone, e.g. acetone; and/or
adding an aqueous solution comprising at least 80 wt %, such as at least 90 or 95 wt %, water.

24. The process according to claim 1, wherein the recovered cellulose pulp or the waste textiles is subject to:
a washing step to remove impurities, such as metals and inorganic salts; and/or
a bleaching step, such as using a bleaching chemicals being at least one of ozone, hydrogen peroxide, peracetic acid or a dithionite, preferably zinc dithionite;
preferably wherein the ISO brightness of the bleached cellulose pulp according to ISO 3688:1999 exceeds 90.

25. The process according to claim 1, wherein the recovered cellulose pulp is saccharified to monosaccharides by acid hydrolysis and/or by enzyme treatment.

26. The process according to claim 25, wherein the acid hydrolysis is performed in the presence of one or more solid acids, sulphonic acids, or mineral acids, such as hydrochloric acid or sulfuric acid, or a combination thereof.

27. The process according to claim 25, wherein the monosaccharides are converted to organic fine chemicals by fermentation, one or more microbial processes and/or catalytic processes, or a combination thereof, such as being converted to 5-Chloromethylfurfural (CMF), caprolactam, 5-(hydroxymethyl)-furfural (HMF), 2,5-Furandicarboxylic acid (FDCA), or adipic acid.

28. The process according to claim 25, wherein one or more acids used in the acid hydrolysis and/or one or more enzymes used in the enzyme treatment are at least partially recycled to be reused for further treatment of cellulose pulp.

29. The process according to claim 1, wherein the cellulose pulp is used as dissolving pulp in the production of regenerated cellulosic fibers.

30. The process according to claim 1, wherein the process further comprises the step of derivatizing the cellulose in the cellulose pulp into dialcohol cellulose, carboxymethyl cellulose, cellulose acetate, or cellulose ethers.

31. The process according to claim 30, wherein cellulose acetate is produced from the cellulose pulp, preferably under homogeneous conditions wherein cellulose is dissolved in an ionic liquid.

32. The process according to claim 1, wherein cellulose acetate is formed by adding at least one of acetic acid and acetic acid anhydride to the solution of cellulose and MeX and subsequently precipitating cellulose acetate.

33. The process according to claim 31, wherein the cellulose acetate is used for spinning a textile fiber.

34. The process according to claim 1, wherein the process for valorization of a crude waste textile material is integrated into a pulping process at a pulp mill, such as a pulp mill designed for production of kraft, chemi-thermomechanical pulp CTM/CMP, thermomechanical pulp, or a mill operating with recycled cellulosic fiber as feedstock.

35. The process according to claim 1, wherein the process for valorization of a crude waste textile material is integrated into a pulping process at a pulp mill designed for production of kraft pulp, and wherein the process further comprises the step of subjecting the precipitated cellulose pulp to an alkaline treatment in the pulp mill.

36. The process according to claim 1, wherein the process for valorization of a crude waste textile material is integrated into a pulping process at a sulfite mill.

37. The process according to claim 36, wherein the process further comprises bleaching the waste textile in a bleaching step in the sulfite mill.
